(19) United States
(12) Patent Application Publication
(10) Pub. No.: US 2006/0204509 A1
(43) Pub. Date: Sep. 14, 2006

(54) Accelerated CD8+ T-cell memory after dendritic cell vaccination

(76) Inventors: John T. Harty, Iowa City, IA (US); Vladimir P. Badovinac, Iowa City, IA (US); Kelly A.N. Messingham, Iowa City, IA (US)

Correspondence Address: NEEDLE & ROSENBERG, P.C., SUITE 1000, 999 PEACHTREE STREET, ATLANTA, GA, 30309-3915, US

(21) Appl. No.: 11/375,653

(22) Filed: Mar. 14, 2006

Related U.S. Application Data
(60) Provisional application No. 60/661,355, filed on Mar. 14, 2005.

(51) Int. Cl. A61K 39/00

(52) U.S. Cl. 424/184.1; 424/93.7

(57) ABSTRACT

Disclosed are compositions and methods for Here, it is shown that vaccination with peptide-coated dendritic cells (DC) generated antigen-(Ag)-specific CD8⁺ T-cells with the phenotype and function of memory T-cells as early as 4-6 days after immunization. These memory-like CD8⁺ T-cells underwent vigorous secondary expansion, resulting in rapid generation of elevated secondary memory CD8+ T-cell numbers and increased protective immunity, in response to a variety of booster immunizations. However, concurrent inflammation prevented the rapid generation of memory T-cells by DC immunization. Thus, DC vaccination, in the absence of overt inflammation, accelerated memory cell generation and dramatically reduced the interval required for booster amplification of memory CD8⁺ T-cell numbers.

This application claims the benefit of U.S. Provisional Application No. 60/661,355, filed on Mar. 14, 2005, which is incorporated by reference herein in its entirety.

This work was supported by NIH grants AI46653 the government has certain rights to this invention.

### I. BACKGROUND

Infection with a pathogenic organism or encounter with a foreign antigen in a host subject causes the subject to mount various humoral and cell-mediated immune responses comprised of T-cells and B-cells (including plasma cells) in an effort to remove the pathogen or foreign antigen. Following exposure, a portion of those T and B-lineage lymphocytes specific for the pathogen or antigen are maintained in the host for many years without further antigenic exposure. This maintenance of specific T and B lympocytes is referred to as immunological memory, the hallmark of which is the maintained ability of the host to mount rapid recall responses upon future antigenic encounter.

The establishment of immunological memory is one of the goals of vaccine development. Yet, the establishment of immunological memory can take months to occur following initial antigenic encounter. Additionally, the mere establishment of immunological memory is not necessarily sufficient to confer protection against future encounters with a pathogen or foreign antigen, as a small memory population may be overwhelmed by a pathogen. Therefore an additional goal is to establish a memory population large enough to provide the protection. For vaccine development, the sufficiency of the immunological memory can be improved through the administration of additional applications of the same or related antigens as the initial vaccine, referred to as a boost. However, multiple boosts are often needed and current immunization regimens often require months between successive vaccine administrations. Thus, a continued problem plaguing vaccine development is the establishment of an effective means to rapidly establish protective immunity.

### II. SUMMARY

Disclosed are methods and compositions related to the rapid establishment of protective immunity.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the accelerated response to booster infection after DC immunization. (A,B) BALB/c mice were infected with virulent L. monocytogenes (1×10³; 0.1 LD₅₀) or immunized with DC-coated with LLO₉₁₋₉₉ peptide (SEQ ID NO: 1) on day 0 and boosted with virulent L. monocytogenes (1×10⁴; 1.0 LD₅₀) at various days after primary immunization. (A) Total number of LLO₉₁₋₉₉-specific CD8⁺ T-cells per spleen on the indicated days after initial L. monocytogenes infection or DC-LLO immunization determined by peptide-stimulated intracellular IFN-65 staining. Data are mean+SD of at least three mice per time point. Arrows indicate the times of booster immunization with L. monocytogenes. (B) Total number of LLO₉₁₋₉₉-specific CD8⁺ T-cells per spleen determined 40 days after the booster infections. Data are mean+SD of three mice per time point. ns, not significant; ** p<0.01. (C through E) Mice were infected with virulent L. monocytogenes (1×10³; 0.1 LD₅₀), or immunized with uncoated (DC-none), or with LLO₉₁₋₉₉ coated (DC-LLO) DC on day 0 and boosted with virulent L. monocytogenes (1×10⁴; 1.0 LD₅₀) at day 6 after primary immunization. Naïve mice were introduced into the experiment at the time of booster infection. (C) Bacterial numbers (mean+SD for 3 mice per group) in the spleen on day 3 and day 5 after booster infection. Only 1 of 3 mice in LM+LM group had detectable bacteria at day 3. LOD=limit of detection. (D) Frequency of LLO₉₁₋₉₉-specific CD8⁺ T-cells from representative mice at the indicated days after initial or booster infection. Numbers represent the percent of IFN-γ⁺CD8⁺ T-cells in the presence (upper number) or absence (lower number) of LLO₉₁₋₉₉ peptide stimulation. (E) Total number per spleen (mean+SD) of LLO₉₁₋₉₉-specific CD8⁺ T-cells obtained from three mice per group per time point.

FIG. 2 shows increased frequencies of memory CD8⁺ T-cells in lymphoid and non-lymphoid tissues in DC+LM mice. BALB/c mice were immunized with virulent L. monocytogenes (1×10³; 0.1 LD₅₀) or DC coated with LLO₉₁₋₉₉ peptide on day 0 and boosted with virulent L. monocytogenes (1×10⁴; 1.0 LD₅₀) on day 6 after primary immunization. The frequencies of LLO₉₁₋₉₉-specific CD8⁺ T-cells were determined by intracellular IFN-g staining in the presence of LLO₉₁₋₉₉ peptide 39 days after the booster infection. Numbers represent the percent of IFN-γ⁺CD8⁺ T-cells in the presence (upper number) or absence (lower number) of LLO₉₁₋₉₉ peptide stimulation. Contour plots from representaive mouse out of two analyzed are shown. PBL, peripheral blood leukocytes; LU, lung; BM, bone marrow; LI, liver; SP, spleen.

FIG. 3 shows that the magnitude of expansion and memory CD8⁺ T-cell numbers in DC immunized mice are determined by dose of L. monocytogenes booster infection. (A) Groups of BALB/c mice were immunized with DC coated with LLO₉₁₋₉₉ peptide on day 0 and were boosted with the indicated doses of virulent L. monocytogenes on day 6 post infection. (B) Frequency of LLO₉₁₋₉₉-specific CD8⁺ T-cells from representative mice at the indicated days post immunization. Numbers represent the percent of IFN-γ^{+CD}8⁺ T-cells in the presence (upper number) or absence (lower number) of LLO₉₁₋₉₉ peptide stimulation. Fold increase is calculated using total numbers of LLO₉₁₋₉₉-specific CD8⁺ T-cells obtained in the spleen from three mice per group 30 days after the booster infection.

FIG. 4 shows that increased numbers of memory phenotype CD8⁺ T-cells in DC+LM mice provide increased protective immunity. (A) BALB/c mice were infected with virulent L. monocytogenes (1×10³; 0.1 LD₅₀) or immunized with LLO₉₁₋₉₉-coated DC on day 0 and boosted with virulent L. monocytogenes (1×10⁴; 1.0 LD₅₀) on day 6 after primary immunization. 68 days after booster infection both groups as well as naïve (control) mice were challenged with high dose of virulent L. monocytogenes (1×10⁶; 100.0 LD₅₀). (B) Frequency of LLO₉₁₋₉₉-specific CD8⁺ T-cells from representative mice at day 6+68. (C) Phenotypic (CD127, CD43 (1B11 mAb), CD44) and functional (TNF, IL-2) status of IFN-γ⁺CD8⁺ T-cells at day 68 post booster infection. (D) Bacterial numbers (mean+SD for 3 mice per group) in the spleen and liver on day 2 after the high dose challenge. LOD=limit of detection. **, all naïve mice died by day 2 after high dose L. monocytogenes challenge. The percent decrease in bacterial numbers in DC-LLO/LM immunized mice compared to LM+LM is indicated. (E) The percent survival at various days after high dose challenge.

FIG. 5 shows amplified secondary memory in DC-peptide immunized mice in response to multiple boosting regimens and against weak antigens. BALB/c mice were infected with (A) VV-LLO, (C, E, G) virulent L. monocytogenes (1×10³; 0.1 LD₅₀), or immunized with (A, C, E) LLO₉₁₋₉₉-coated DC, or (G) p60₄₄₉₋₄₅₇ (SEQ ID NO: 2)-coated DC on day 0. On day 6 mice were boosted with (A) VV-LLO, (C) attenuated actA-deficient L. monocytogenes (1×10^{7; 1.0} LD₅₀), (E) LLO₉₁₋₉₉ coated naïve splenocytes (2×10⁷/mouse), or (G) virulent L. monocytogenes (1×10⁴; 1.0 LD₅₀). (B, D, F) Total number per spleen (mean+SD) of LLO₉₁₋₉₉-specific, or (H) p60₄₄₉₋₄₅₇-specific CD8⁺ T-cells obtained from three mice per group per time point. Numbers inside panels indicate fold increase in total numbers of Ag-specific CD8⁺ T-cells in mice initially immunized with peptide coated DCs compared to other initial immunizations.

FIG. 6 shows increased MHC class Ib CD8⁺ T-cell response after early booster infection of DC-fMIGWII (SEQ ID NO: 3) immunized mice. FIG. 8A shows that BALB/c mice were immunized with virulent L. monocytogenes (1×10³; 0.1 LD₅₀) or DC coated with the H2-M3 (class Ib) restricted f-MIGWII epitope from LM on day 0 and boosted with virulent L. monocytogenes (1×10⁴; 1.0 LD₅₀) on day 6 after primary immunization. FIG. 6B shows the frequencies of f-MIGWII and LLO₉₁₋₉₉-specific CD8⁺ T-cells from representative mice at the indicated days post immunization. Numbers represent the percent of IFN-γ⁺CD8⁺ T-cells in the presence (upper number) or absence (lower number) of LLO₉₁₋₉₉ peptide stimulation. FIG. 6C shows the total number (mean+SD of three mice per group) of f-MIGWII and LLO₉₁₋₉₉-specific CD8⁺ T-cells in the spleen 41 days after booster infection. LOD=limit of detection.

FIG. 7 shows that Ag-specific CD8⁺ T-cells exhibit phenotypic and functional characteristics of memory T-cells early after DC-peptide immunization. (A,B) BALB/c mice were infected with virulent L. monocytogenes (0.1 LD₅₀) or LLO₉₁₋₉₉-coated DC and on day 6 post immunizations the LLO91-99-specific CD8⁺ T-cells in the spleen were detected with (A) tetrameric MHC class I-LLO₉₁₋₉₉-complexes or with (B) intracellular IFN-γ staining in the presence of LLO₉₁-₉₉ peptide stimulation. (A) Thin line represents the isotype control staining, thick line represents staining with mAbs of the indicated specificity of gated tetramer positive cells from representative mice. Numbers represent the % of cells positive for the indicated molecules. FIG. 7B shows TNF and IL-2 production by IFN-γ⁺CD8⁺ T-cells after in vitro stimulation with LLO₉₁₋₉₉ peptide. Upper numbers represent the percent of TNF (or IL-2)⁺IFN-γ⁺CD8⁺ T-cells. Lower numbers represent the background staining with isotype control Abs after peptide stimulation. Data are representative of three to six mice. FIG. 7C shows purified naïve OT-I Thy1.1 cells were transferred into naive C57BL/6 Thy1.2 mice and one day later mice were immunized with actA-deficient LM-OVA (1×10⁶) or OVA₂₅₇₋₂₆₄ (SEQ ID NO: 6)-coated DC and on the indicated days after immunization the CD8⁺/Thy1.1⁺ cells in the spleens were analyzed. Non-immunized recipient mice were used as controls (day 0). Results are presented as % of CD8⁺/Thy1.1⁺ cells that were positive for indicated molecules. % of IL-2 producing CD8⁺/Thy1.1⁺ cells was determined after in vitro stimulation with OVA₂₅₇₋₂₆₄ peptide. Data are presented as mean±SD for three to five mice per group. Data are representative of three independent experiments.

FIG. 8 shows rapid memory CD8⁺ T-cell generation and vigorous secondary expansion after booster-infection of DC-immunized LM-immune hosts. FIG. 8A shows that Naïve or LM-immune (d75 after infection with 1×10⁶ actA-deficient LM) BALB/c mice were immunized with NP₁₁₈₋₁₂₆-coated-DC (DC-NP) and boosted at d5 with virulent LM expressing NP₁₁₈₋₁₂₆ (LM-NP; 1×10⁴). FIG. 8B shows the phenotypic and functional status of NP₁₁₈₋₁₂₆- or LLO₉₁₋₉₉-specific CD8⁺ T-cells at d5 post DC-NP immunization. FIG. 8C shows the phenotypic and functional status of LLO₉₁₋₉₉-specific CD8⁺ T-cells at d6 post virulent LM-infection (1×10³) of naïve mice. FIG. 8D shows the frequency of NP₁₁₈-126- (SEQ ID NO: 7) or LLO₉₁₋₉₉-specific CD8⁺ T-cells from representative mice at the indicated days after initial (d5) and booster (d5+5) immunizations. FIG. 8E shows the total number/spleen (mean+SD, 3 mice/group) of NP₁₁₈₋₁₂₆-, LLO₉₁₋₉₉- or p60₂₁₇₋₂₂₅-specific CD8⁺ T-cells. Fold-increase in total numbers of NP118-126-specific CD8⁺ T-cells at d5 after booster immunization is indicated.

FIG. 9 shows that DC-peptide immunization accelerates the transition of CD8⁺ T-cells from an effector to memory phenotype. Purified naïve OT-I cells (Thy1.1) were transferred into naive C57BL/6 mice (Thy1.2) and one day later mice were immunized with actA-deficient LM-OVA (0.1 LD₅₀) or OVA₂₅₇₋₂₆₄-coated DC and on the indicated days after immunization the CD8⁺/Thy1.1⁺ cells in the spleens were analyzed. Non-immunized recipient mice were used as controls. (a) Shaded histogram represents the isotype control staining, thick line represents staining with mAbs of the indicated specificity of Thy1.1⁺/CD8⁺ T-cells from representative mice. Numbers represent the % of cells positive for the indicated molecules. (b) IL-2 production by IFN-γ⁺CD8⁺ T-cells after in vitro stimulation with OVA₂₅₇₋₂₆₄ peptide. Numbers represent the percent of IL-2 positive Thy1.1⁺/CD8⁺ T-cells.

FIG. 10 shows that inflammation controls the accelerated secondary response of Ag-specific CD8⁺ T-cells after DC immunization. (A) BALB/c mice were immunized either with the L. monocytogenes LLO92F strain that lacks a functional LLO₉₁₋₉₉ epitope (1×10³), DC coated with LLO₉₁₋₉₉ peptide (DC-LLO), or both (LM LLO92F+DC-LLO) and all groups of mice were boosted with the virulent, LLO₉₁₋₉₉ expressing L. monocytogenes strain (1×10⁴; 1.0 LD₅₀) on day 6 after primary immunization. Total number (mean+SD of three mice per group) of LLO₉₁₋₉₉- and p60₂₁₇₋₂₂₅ (SEQ ID NO: 4)-specific CD8⁺ T-cells in spleen at the indicated days after infection The numbers inside the panels indicate fold increase in total numbers of LLO₉₁₋₉₉-specific CD8⁺ T-cells. BLD, below limit of detection. (B through E) BALB/c mice were immunized with DC-LLO alone (w/o CpG group) or in combination with CpG ODNs (w/CpG) and both groups of mice were boosted with the virulent LM (1×10⁴; 1.0 LD₅₀) on day 6 after primary immunization. One representative experiment out of two is shown. (B) % of LLO₉₁₋₉₉-specific CD8⁺ T-cells detected by ICS for IFN-γ that were positive for CD127, CD43 and IL-2. Data are presented as mean+SD of three mice per group. (C) Bacterial numbers (mean+SD for two to three mice per group) in the spleen and liver on day 2 after the LM challenge. Naive mice that were not immunized on day 0 were used as controls. (D) The frequencies of LLO₉₁₋₉₉-specific CD8⁺ T-cells were determined by intracellular IFN-g staining in the presence of LLO₉₁₋₉₉ peptide 6 days after the booster infection. Numbers represent the percent of IFN-γ⁺CD8⁺ T-cells in the presence (upper number) or absence (lower number) of LLO₉₁₋₉₉ peptide stimulation. Contour plots from one representative mouse out of three analyzed are shown. (E) Total number (mean+SD of three mice per group) of LLO₉₁₋₉₉-specific CD8⁺ T-cells in spleen at the indicated days after infection The numbers inside the panels indicate fold increase in total numbers of LLO₉₁₋₉₉-specific CD8⁺ T-cells. One representative experiment out of two is shown.

FIG. 11 shows that inflammation prevents accelerated generation of memory CD8⁺ T-cells and early prime-boost. (a-c) BALB/c mice received DC-LLO alone (w/o CpG group) or with CpG (w/CpG) and were boosted with LM (1×10⁴) on d6. One of four experiments is shown. (a) Total number and fold-increase of LLO₉₁₋₉₉-specific T-cells in spleen at the indicated days. (b) Bacterial numbers in organs on d2 after boost. (c) Percent of LLO₉₁₋₉₉-specific T-cells expressing CD127, CD43 and IL-2. (d) C57BL/6 (Thy1.2) mice received no immunization, DC-OVA alone (w/o CpG group) or with CpG (w/CpG) and were injected with naïve OT-I cells (Thy1.1, 5×10⁵) on the indicated days. Results are the frequency of OT-I Thy1.1 cells in spleens at d3 after injection. (e-f) BALB/c mice received DC-LLO alone (w/o CpG) or with CpG on d0 (w/CpG d0) or d3 (w/CpG d3) and all mice were boosted with virulent LM (1×10⁴) on d7. One of two experiments is shown. (e) Phenotypic and functional status of LLO₉₁₋₉₉-specific T-cells at d7 post DC-LLO immunization. (f) Total number and fold-increase of LLO91-99-specific T-cells in spleen. (g) C57BL/6 (WT) and IFN-γRII^{-/-} mice were immunized with DC-OVA alone (w/o CpG) or with CpG (w/CpG) and were boosted with actA-deficient LM-OVA (5×10⁶) on d6. Total number and fold-increase of OVA₂₅₇₋₂₆₄-specific T-cells in spleen. Data in a,b,c,d,f and g are mean+SD, 3 mice/group.

FIG. 12 shows the in vitro maturation of DC with CpG does not prevent rapid memory CD8⁺ T-cell generation in vivo. Naïve BALB/c mice were immunized with LLO₉₁₋₉₉ peptide coated DCs that were matured in the presence of LPS, CpG (10 μg/ml), or LPS+CpG for the last 24 hours of an in vitro culture. All groups of mice were boosted with virulent L. monocytogenes (1×10⁴; 1.0 LD₅₀) on d7 after primary DC-LLO immunization. FIG. 12A shows the frequency of LLO₉₁₋₉₉-specific CD8⁺ T-cells from representative mice at d7 after DC-immunization. Numbers represent the percent of IFN-γ⁺CD8⁺ T-cells in the presence (upper number) or absence (lower number) of LLO91-99 peptide stimulation. FIG. 12B shows phenotypic (CD127, CD27, CD43) and functional (TNF, IL-2) status of LLO91-99-specific IFN-γ⁺CD8⁺ T-cells at d5 post DC-LLO immunization. FIG. 12C shows the percentage of LLO91-99-specific CD8⁺ T-cells detected by ICS for IFNγ that were positive for CD127, CD27, CD43, TNF and IL-2. Data are presented as mean+SD of three mice per group. FIG. 12D shows the frequency of LLO₉₁₋₉₉-specific CD8⁺ T-cells from representative mice at d7+5 after DC and LM immunizations. FIG. 12E shows the total number per spleen (mean+SD) of LLO₉₁₋₉₉-specific CD8⁺ T-cells obtained from three mice per group per time point.

FIG. 13 shows that BALB/c mice were immunized with LPS-matured dendritic cells (DC) coated with the AH1/AH5 peptide from the CT26 colon carcinoma tumor. On day 6 after DC immunization, mice were boosted with attenuated Listeria monocytogenes expressing the AH1/AH5 epitope. FIG. 13A shows the frequency of AH1/AH5 specific CD8 T cells in the spleen from representative mice at d6 after DC immunization (left panel) or d6 after boosting. The number in parenthesis is the background from the unstimulated sample. FIG. 13B shows the total number of AH1/AH5-specific CD8 T cells/spleen from 3 mice/group at the same time points as in (a). The number represents the fold increase in boosted mice compared to unboosted mice.

FIG. 14 shows that C57B1/6 mice were immunized with LPS-matured dendritic cells (DC) coated with the (A, B) Trp1 or Trp2 (C,D) peptide from the B16 melanoma tumor. On day 6 after DC immunization, mice were boosted with attenuated Listeria monocytogenes expressing the Trp1 or Trp2 epitope. Frequency of Trp1 (A) or Trp2 (C) specific CD8 T cells in the spleen from representative mice at d6 after DC immunization (top) or d6 after boosting (bottom). The number in parenthesis is the background from the unstimulated sample. Total number of Trp1 (B) or Trp2 (D)-specific CD8 T cells/spleen from 3 mice/group at the same time points as in (A, C). The number represents the fold increase in boosted mice compared to unboosted mice.

FIG. 15 shows that BALB/c mice were immunized with LACK-peptide coated DC. On day 7, these mice and naïve mice were boosted with LM-LACK. FIG. 15A shows the frequency of LACK specific CD4 T cells detected by peptide stimulated ICS (Top Number, peptide stimulated sample, bottom number, unstimulated background control) from representative mice at the indicated days. FIG. 15B shows the mean+SD from three mice/group/time point.

FIG. 16 shows that BALB/c mice were immunized with DC/Pb9 peptide and the frequency of Pb9-specific CD8 T cells was determined by peptide-stimulated intracellular cytokine staining at d7 (left panels). The remaining mice were boosted with LM-Pb9 and the frequency of Pb9-specific CD8 T cells was determined by peptide-stimulated intracellular cytokine staining at d7+5 (right panels).

FIG. 17 shows the total number of malaria specific CD8 T cells/spleen at the indicated days post immunization in mice that received DC/Pb9 (squares), LM-Pb9 (upright triangles) or DC/Pb9+LM-Pb9 booster at d7 after DC immunization (upside down triangles). Two malaria challenge experiments were carried out at d21 and d28 after boost. 100% of DC/LM-Pb9 mice at each day (12/12 and 12/12) were completely protected from the appearance of blood stage malaria, while 100% of control, non-vaccinated mice (12/12 and 12/12) developed blood parasitemia by day 6 post challenge. LM-Pb9 vaccination alone afforded no protection while DC/Pb9 alone afforded 50% protection from challenge.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. Also each number between the ranges is also disclosed, such as, 10, 11, 12, 13, 14, land 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF MAKING THE COMPOSITIONS

The compositions disclosed herein and the compositions necessary to perform the disclosed methods can be made using any method known to those of skill in the art for that particular reagent or compound unless otherwise specifically noted.

Following exposure to a target foreign antigen or pathogen, the immune system works to remove the target through the generation of T-cells and B-cells specific to the target. For B-cells this involves the binding of the B-cell receptor to the target and ultimately the evolution of the target specific B-cells into plasma cells which secrete antibody specific for the target. For T-cells, the process is slightly more complex. Unlike B-cells which directly recognize the target, T-cells can only recognize peptides presented to the T-cell receptor in the context of a major histocompatibility complex (MHC) molecule. To achieve this scenario, the target must be internalized by a cell capable of presenting antigen in the context of MHC, an antigen-presenting cell, for example, a dendritic cell. Once the target is internalized, the cell breaks the target into small peptides which are combined with the cells MHC molecule and presented on the surface. Naïve T-cells with a T-cell receptor (TCR) specific for the peptide-MHC combination can then recognize the target and become “activated” upon the binding of the TCR to the MHC-peptide being presented on the antigen presenting cell. Once activated the naïve T-cell, now referred to as an “effector cell,” is characterized as having one or more of the following markers: CD44⁺(positive), CD11a⁺(positive), CD62L^{lo}, CD69⁺(positive), Bcl-2^{lo}, CD27^{lo}, and CD127⁻(negative). Additionally, the effector cell is capable of rapid proliferation. Typically the effector cells begin dividing within 24 hours of the initial stimulation and can possess doubling times of 6-8 hrs per division. The effector cells also start to produce cytokines such as IFN-65 and TNF-α, as well as, the production of cytolytic agents such as perforin and granzyme B. The generation of target specific T-cells follows three primary phases. The first phase is an expansion phase. Here, the target specific T-cells rapidly proliferate and are composed predominantly (>95%) of effector cells. Within one to two weeks, the expansion of the effector T-cells reaches a maximum and the second phase, a contraction phase begins. During the contraction phase approximately 90-95%, for example, of target specific T-cells die. Those target specific cells that do not die initiate the memory pool and this population is maintained at stable numbers maintained as long-lived target specific T-cells for extended periods of time. Thus, begins the third phase, a steady-state phase called immunological memory.

The establishment of a long-lived immune response to a target that is of a size large enough to protect the recipient and generated quickly enough to meet the needs of those receiving a vaccine is the continuing goal in the development of many vaccines. Vaccines refer to any composition that is administered to a subject with the goal of establishing an immune response to a particular target or targets. In certain embodiments the vaccines will produce an immune response that is a protective immune response. Vaccines can be, for example, prophylactic, that is, administered before a target is ever encountered, as is typically the case for Polio, measles, mumps, rubella, smallpox, chicken pox, and influenza vaccines, for example. Vaccines can also be therapeutic, providing an immune response to a target that is already within a subject, for example, a vaccine to a particular cancer. Typically vaccines are administered in a single or multiple doses called immunizations and are designed to generate memory T and B-cell populations. However, to date, no vaccine designed to generate memory T-cells has accomplished this task with a single dose, or immunization, of the vaccine. Often with vaccines directed to T-cell immunity, the initial immunization, or prime, generates a memory T-cell population that is insufficient to provide protection against future target encounter related to the antigen. Additionally, the few memory T-cells that are generated from the initial prime can take at least 2 months and can take years to finally transform from naïve T-cells into memory T-cells. To overcome the problem of inadequate initial priming, additional immunizations, or boosts, comprising the same or related antigen are used to bolster the numbers of memory T-cells. However, for a boost to be effective, the memory T-cell population must be stabilized. That is, the target-specific T-cell population must have completed the transformation to memory cells and be in a steady-state. Thus, a prime-boost immunization regimen can require months between immunizations creating a tremendous lag in time between when immunity to a target is desired and when it is actually achieved. The methods disclosed herein overcome these problems. Disclosed herein are compositions and methods that increase the number of memory T-cells produced after an initial immunization and/or which decrease the time within which these memory T-cells are generated. Furthermore, the compositions and methods can also decrease the number of boosts that are needed to achieve protective immunity, as well as decreasing the time needed between the initial immunization and the first boost as well as the time needed between the first or subsequent boost and other subsequent boosts. Also, disclosed herein are methods of producing memory T-cells specific for a target in a subject comprising administering to the subject a mixture comprising an antigen related to the target and a dendritic cell, and administering a booster to the subject within one week of initial antigen contact, and wherein the memory T-cells generated are able to proliferate upon encounter with the booster. Also, disclosed herein are methods of producing memory T-cells specific for a target in a subject comprising administering to the subject a mixture comprising an antigen related to the target and a dendritic cell, and administering a booster to the subject less than 6 months after initial antigen contact, and wherein the memory T-cells generated are able to proliferate upon encounter with the booster.

Also disclosed are methods, wherein the booster is administered less than 5 months, 4 months, 3 months, 2 months, 1 month, 4 weeks, 3 weeks, 2 weeks, 13 days, 12 days, 11 days, 10 days, 9 days, 8 days, 7 days, 6 days, 5 days, 4 days, 3 days, 2 days, or 1 day after initial antigen contact.

Typically, memory T-cells can be characterized as long-lived antigen-specific T-cells having a combination of two or more of the following markers CD44⁺ (positive), CD11a⁺ (positive), CD43^{1B11-}(negative), CD62L^{HI or LO}, CD127⁺(positive), and CD45RA⁻ (negative), CD27^{hi}, CD122^{hi}, IL-15R+. Memory T-cells can be divided into two major groups distinguished by the expression of CCR7 and CD62L. CCR7⁻, CD62L^{lo} (negative) memory T-cells are referred to as “effector memory T-cells” (T_{EM}). These cells generally are localized in the peripheral tissues such as the liver and lungs as well as the spleen, and produce rapid effector functions, such as IFN-65 production, upon stimulation. CCR7⁺ (positive) memory T-cells generally localize in the secondary lymphoid organs such as the thymus, bone marrow, and lymph nodes, although they can also be found in peripheral tissues. These cells are referred to as “central memory T-cells” (T_{CM}) and provide more effective protection to the host, against at least some pathogens, through increased proliferative capacity. It is understood that maintained within a population of memory T-cells is the potential for further expansion upon future antigen encounter. Thus, herein disclosed are methods of generating memory T-cells. The memory T-cells can be generated, for example, by mixing a target or antigen related to the target with dendritic cells and administering the mixture to a subject. It is understood that the disclosed methods can be used for the generation of, for example, central memory T-cells. Thus, also disclosed are methods of generating an immune response to a target in a subject comprising mixing the target or an antigen related to the target with dendritic cells and administering the mixture to the subject, wherein the mixture increases the number of central memory T-cells. It is understood and herein contemplated that by increasing memory T-cells, a population of central memory T-cells can be generated with sufficient number to confer protection against future encounters with a target. Therefore, also disclosed are methods of generating a protective amount of central memory T-cells in a subject to multiple antigens comprising mixing dendritic cells with the antigens and administering the mixture to the subject, wherein the protective amount of central memory T-cells are generated more quickly than are generated with the antigen alone. For example, specifically contemplated are methods wherein a protective amount of central memory T-cells are generated in 6, 12, 18, 24, or 30 days.

Herein, “immunological memory” refers to the physiological condition characterized by long-lived antigen-specific lymphocytes with the ability to provide rapid recall responses upon future antigen experience. It is understood and herein contemplated that the lymphocytes that provide this protection can be CD4 or CD8 T-cells specific for the antigen.

It is understood and herein contemplated that disclosed methods of generating memory T-cells can provide a booster immunization within one week of the initial antigen contact. Thus, for example, a booster may be given 1, 2, 3, 4, 5, 6, or 7 days following the initial antigen contact. It is also understood that the booster may be given, prior to 6 months, 5 months, 4 months, 3 months, 2 months, 1 month, or 2 weeks after the initial antigen contact.

It is understood that the memory T-cells generated in response to the administration of antigen in combination with dendritic cells do not require a refractory period, or rest, before a boost is given. By “refractory period” is meant a period of time with limited or no antigen contact after the antigen-specific T-cells are generated to allow for the development of memory cells. Thus, it is not required that a steady state be reached within the population of target specific T-cells before a boost is given, nor is there need to wait for the specific T-cell response to contract from its peak number during the effector response (i.e. after the expansion and contraction phases). Administration of the booster can occur at any point during the initial expansion of the effector response up to any number of days prior to 12 months following initial antigen exposure. For immunological responses that take longer than one week to reach the peak of the expansion phase, such as responses to chronic infections or parasitic infections, the period for which the booster may be administered can be further drawn out to at least the day maximum expansion of target specific T-cells is reached. One of skill in the art would readily know the time needed for maximal expansion to occur for a given target and thus will easily be able to determine if the booster can be administered 6, 7, 8, 9, 10, 11, 12, 13, 14, 15, or 20 days following initial antigen exposure.

It is also contemplated that the booster immunization can comprise any antigen related to the target including, but not limited to, the same antigen supplied in the mixture provided in the prime comprising an antigen related to the target and a dendritic cell. Thus, it is understood that the antigen provided in the booster can be different from the antigen in the prime. For example, the antigen used in the mixture used in the prime can be a peptide related to the target while the boost can be a live-attenuated strain of the target. It is also understood that the disclosed methods can comprise more than one boost. Thus, for example, disclosed are methods of producing memory T-cells specific for a target in a subject comprising administering to the subject a mixture comprising an antigen related to the target and a dendritic cell, and administering a booster to the subject within one week of initial antigen contact, and administering a second booster to the subject 30, 31, 32, 33, 34, 35, 36, 37, 38, 39, 40, 41, 43, 43, 44, 45, 46, 47, 48, 49 50, 55, 60, 70, 80, 90, 100, 120, or 180 days (or any number of days in between) following the first boost, wherein the memory T-cells generated are able to proliferate upon encounter with each booster.

Additionally, it is understood that the booster can comprise, in addition to the antigen any eukaryotic cell type able to present that antigen to T-cells ( for example, an antigen presenting cell such as a B cell, a dendritic cell, T-cell, macrophage, as well as any other nucleated cell that presents MHC class I on its cell surface.). Thus, it is also understood that the booster can further comprise a nucleated cell that presents MHC class I. Typical examples of nucleated cells that present MHC class I, include but are not limited to splenocytes, dendritic cells, peripheral blood lymphocytes, fibroblasts, macrophages, B cells, irradiated or inactivated tumor cells. The purpose of the administration of the boost is to increase the number of T-cells specific to the target. It is understood and herein contemplated that the boost has the effect of stimulating increased numbers of memory T-cells and effector T-cells specific to the target. For example, following a boost, the numbers of effector T-cells specific for the target can be increased 5-300-fold within five days of immunization or booster administration. Thus, for example, specifically disclosed are methods whereby the initial DC administration is followed within one week by a booster administration leading to 5-300-fold increases in the number of antigen-specific effector CD8+ T-cells within five days after booster administration. Similarly, the numbers of memory T-cells can increase 3-30-fold compared to DC vaccination alone and within 30 days after the initial administration.

Disclosed herein, the mixture comprising an antigen related to the target and a dendritic cell can stimulate the production of memory T-cells as well as effector T-cells. It is also disclosed that the number of these cells produced is larger than prime-boost regimens that do not use a mixture of antigen and a dendritic cell in the prime. It is understood and herein contemplated that the number of memory T-cells generated by the administration of the mixture of an antigen related to a target and a dendritic cell can be sufficient to provide protective immunity without further administration of the antigen in the form of a boost. It is also understood that the memory T-cell generation occurs within one week and thus immune protection can be conferred within one week. It is understood and herein contemplated that immune protection can occur in 3, 4, 5, 6, or 7 days. The rapid generation of protective immunity is beneficial to those seeking the establishment of protective immunity in an accelerated way and would have particular application in the protection of individuals exposed to biological agents or diseases, terminally ill patients seeking therapeutic vaccinations (e.g., a cancer patient), individuals who are traveling on short notice and need immunity prior to exposure, or as a defense against bio-warfare or bio-terrorism. Thus specifically disclosed are methods of producing protective immunity to a target in a subject, comprising administering a mixture comprising an antigen related to the target and a dendritic cell, wherein the protective immunity is generated within one week, and wherein the subject has sought to achieve protective immunity in an accelerated way.

Herein, “protection” and “protective immune response” refer to an immune response that is able to reduce the severity of an antigenic insult or pathogen. It is understood that immunological memory can occur with protection, but protection cannot occur without immunological memory. Such responses can include, but are not limited to a reduction or the complete ablation of all symptoms associated with a future antigenic insult or pathogen encounter. For example, a protective immune response to reduce the severity of an infection with a pathogen by 10%, 20%, 30%, 40%, 50%, 60%, 70%, 80%, 90%, or 100%, a 100% reduction representing the prevention of the establishment of an infection. Similarly, for chronic infection, the prevention of the establishment of a chronic infection, would represent a reduction in the severity of the disease even though an acute infection may still result. Protective immunity is also understood to occur when future encounters with a pathogen that causes an acute infection are reduced in duration or severity due to the presence of specific immunity. Thus, for example, an acute infection that typically lasts 10 days can be reduced to 4 days due to immuno-protection. Additionally, protection can refer to the loss of lethality of an otherwise lethal infection.

Herein “specificity” or “specific” refers to the selective nature of an acquired immune response, wherein the acquired immune response binds the antigen with a higher affinity than serum albumin. It is understood and herein contemplated that individual T and B lymphocytes do not respond to every antigen presented to them, but only those for which their respective receptors have affinity. For T lymphocytes it is understood that T-cells recognize peptide antigen only in the context of an MHC molecule and then only if the residues presented by the peptide/MHC combination have affinity for particular residues of the T-cell receptor. One of skill in the art can readily identify peptides that are capable of being recognized by a given T-cell. Thus, it is understood that a memory T-cell specific for a target refers to only those T-cells that are capable of generating an immune response to the target and not all memory T-cells.

The disclosed methods of generating T-cells and/or protection are always focused on generating immune response directed against a “target.” Herein, “target” refers to any antigen or pathogen against which an immune response is desired. It is understood that a “target” can comprise a peptide, polypeptide, protein, cell, or organism. Thus, for example, a target can comprise a peptide such as LLO₉₁₋₉₉, (SEQ ID NO: 1) the gag gene of HIV-1, muc-1, or a pathogen such as Listeria monocytogenes. Thus, it is understood and herein contemplated that “target” can refer to any known antigen or pathogen and is not limited to those disclosed herein.

Herein “antigen,” and “foreign antigen,” refer to any molecule capable of generating an immune response, such as a peptide, polypeptide, protein, cell, cancer, live-attenuated pathogen, or heat-killed pathogen that has the potential to stimulate an immune response. Associated with a reference to a foreign antigen, the term “antigenic insult” can be used. It is understood that antigenic insult refers to the effect the foreign antigen has on the subject that stimulates an immune response or a self-antigen associated with a cancer. Additionally, it is understood that “pathogen” or “pathogenic organism” refers to any organism capable of eliciting an immune response from a subject upon infection of the subject with pathogen. It is understood that a given pathogen can be comprised of multiple antigens to which the subject's immune response may respond. It is also understood and herein disclosed that “pathogen” can refer to a virus, bacteria, or parasite, for example. Thus, for example, pathogen can refer to a virus wherein the virus is selected from the group of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

Also disclosed are compositions wherein the pathogen is a bacteria selected from the group of bacteria consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are compositions wherein the pathogen is a parasite selected from the group of parasites consisting of Toxoplasma gondii, Plasmodium falciparum, Plasmodium vivax, Plasmodium malariae, Plasmodium bergheii, other Plasmodium species., Trypanosoma brucei, Trypanosoma cruzi, Leishmania major, Leishmania. donnovani, other Leishmania species., Theileria parva, Schistosoma mansoni, other Schistosoma species., and Entamoeba histolytica.

Herein a “related antigen” refers to any antigen that is derived from the target or possesses significant enough identity to a fragment of the target as to be able to stimulate a specific immune response against the target and the related antigen. Thus, for example, a related antigen can refer to a bacterial protein of the target. Related antigen can also refer to peptides generated in a laboratory that mimic known T-cell epitopes of the target, but are modified to increase their immunogenicity. One of skill in the art will understand what modifications can be made to antigens without destroying the specificity for the target.

“Dendritic cell” or “DC”, as used herein, refers to a mature antigen presenting cell, which is identified by the expression of one or more of the following markers on its cell surface: CD1a, CD1b, and CD1c, CD4, CD11c, CD33, CD40, CD80, CD86, CD83 and HLA-DR. A dendritic cell progenitor means a hematopoietic cell identified by the expression of one or more of the following markers on its cell surface: CD123, CD45RA, CD36, and CD4. “Dendritic cell progenitor”, “dendritic progenitor cells”, “dendritic precursor cell”, “dendritic cell precursor”, can be used interchangeably herein. Furthermore, methods where a dendritic cell can be used can also be accomplished by using a dendritic cell precursor. The dendritic cells or dendritic cell precursors used in the disclosed methods can include but are not limited to dendritic cells from the subject which will later receive the mixture of antigen and dendritic cells. Thus specifically contemplated are methods of producing memory T-cells, wherein the dendritic cells originated from the subject to be vaccinated.

Herein “proliferation” or “expansion” refers to the ability of a cell or population of cells to increase in number.

Herein, “major histocompatibility complex” or “MHC” refers to the cluster of genes that encode MHC molecules. MHC molecules are divided into two classes MHC class I and MHC class II based on the resulting structure of the molecule. MHC class I molecules consist of an alpha chain which folds into 3 alpha domins (a1, a2, and a3) and is stabilized by the presence of b2-microglobulin. When MHC class I molecules present antigen, the antigen is presented in the binding cleft between the a1 and a2 domains as a peptide typically 8 to 10 amino acids long. The length of the peptide is restricted by the closed ends of the binding cleft. Typically, MHC class I molecules present peptide antigens to CD8 T-cells.

MHC class II molecules consist as a dimeric molecule with an alpha chain and a beta chain. Both the alpha chain and the beta chain have two domains. Thus the alpha chain has an a1 and a2 domain and the beta chain has a b1 and b2 domain. For MHC class II molecules, antigen is presented between the a1 and b1 domains. Because the peptide binding cleft is formed by two separate chains, the peptide bound by the MHC class II molecule is not restricted by length though T-cells will only recognize that portion of the peptide presented within the biding cleft. The peptide presented in the context of MHC class II molecules is typically recognized by CD4 T-cells.

The structure of the major histocompatibility complex within each individual comprises multiple genes and multiple alleles for each gene. This allows for great heterogeneity among individuals. Within the human genome, there are multiple class I genes, A, B, C, E, F, G, H, J, and X and 4 class II genes (DP, DM, DQ, and DR). Each gene has multiple alleles that can be expressed. Within mice a similar structure is observed. Mice typically have 3 class I genes K, D, and L, and 3 class II genes A, E, and M. To distinguish the various genes and alleles within individuals, the MHC genotype is referred to as Human Leukocyte Antigen or HLA in humans and simply H-2 in mice. Thus, for example, an individual could have the gene HLA-A2 meaning they have the second allele of the A gene. Similarly, a mouse can be for example Ld positive. As multiple genes and alleles can be expressed on an individual, this allows for the ability for a single human subject to be, for example HLA-A2, A11, B44, and C7. Typically some MHC alleles are more prevalent within a given population. For example, within North America and European Caucasian populations, MHC class I alleles A1, A2, A3, A11, B44, Cw4, and Cw7.

Due to MHC restriction, it is understood and herein contemplated that the same single dendritic cell will not be effective in the disclosed methods for all people. One way to insure compatibility between the recipient and the dendritic cell provided in the mixture is to take the dendritic cell directly from the subject. Thus, herein disclosed are methods of producing memory T-cells specific for a target in a subject comprising administering to the subject a mixture comprising an antigen related to the target and a dendritic cell, and administering a booster to the subject within one week of initial antigen contact, wherein the memory T-cells generated are able to proliferate upon encounter with the booster, and wherein the dendrtitic cells originated from the subject to be vaccinated. Also disclosed is a method of making a vaccine specific for a subject in need thereof comprising removing dendritic cells from the subject to be vaccinated, mixing an antigen with the dendritic cells, administering the mixture to the subject. Alternatively, it is possible to produce multiple versions where the dendritic cells used comprise the common MHC alleles for a population. For example, it is possible to isolate or manufacture a dendritic cell comprising the common MHC alleles for individuals of European dissent. Though not every individual given the vaccine would have all the alleles, many would have at least one and would benefit from the vaccine. However, people without any of these alleles would still receive no benefit beyond the simple administration of the antigen. Thus, though not everyone would benefit from a specific vaccine, many would and the manufacture of multiple versions of a vaccine to cover multiple populations would insure global coverage. Therefore disclosed herein is a composition comprising dendritic cells and one or more antigens, wherein the dendritic cells and antigen are in sufficient quantity to induce a protective immune response more quickly and with greater magnitude then antigen alone, wherein the dendritic cells comprise the common MHC alleles for a given population, and wherein the antigen comprise immunodominant peptides corresponding to the MHC alleles.

Typically, immunodominant epitopes/peptides refers to the epitope and corresponding peptide that constitute the majority of the immune response for a given MHC class and MHC phenotype. For example in an H-2^{d} mouse infected with Lymphocytic choriomeningitis virus (LCMV), the immunodominannt epitope is the Ld epitope NP₁₁₈₋₁₂₆. The remaining epitopes GP₉₉₋₁₀₈ and GP₂₈₃₋₂₉₁ (both K^{d}) are considered to be subdominant. Another example is the infection of an H-2b mouse with LCMV. Here, the D^{b} epitopes GP₃₃₋₄₁, NP396-404, and GP₂₇₆₋₃₀₆ along with the epitope GP₃₄₋₄₃ are considered to be immunodominant. Other peptides such as NP205-212 and GP₉₂₋₁₀₁ are subdominant epitopes. Thus as seen in the H-2d mouse there can be multiple immunodominant epitopes.

Herein, “vaccine” refers to any composition comprising a fragment of one or more antigens or whole antigens wherein the composition stimulates an immune response to the antigen or antigens of the composition. Thus, a vaccine refers to any composition that is administered to a subject with the goal of establishing an immune response to a particular target or targets. Thus, for example, a typical vaccine can comprise a heat-killed virus. Another example of a vaccine is an attenuated strain of the pathogen such as found in the BCG vaccine for M. Tuberculosis. Typically formulations of vaccines can be used throughout and it is understood that the antigens administered can be provided in the context of a vector or DNA immunization. It is also understood that the vaccine compositions can comprise other substances designed to increase the ability of the vaccine to generate an immune response. For example, a typical vaccine can comprise an antigen plus an adjuvant, such as alum, or a cell that enhances antigen presentation such as the dendritic cells disclosed herein. It is also understood that the vaccines disclosed herein can be therapeutic or prophylactic. Thus, for example, the vaccines disclosed herein can be used to prevent an infection such as Listeria monocytogenes, or HIV. Alternatively, the vaccines disclosed herein can be used therapeutically to treat an individual with cancer or a chronic infection such as HIV or HSV-1.

It is understood and herein contemplated that more than one antigen can be provided in the mixture of the disclosed method. For example, a mixture can comprise a peptide for one T-cell epitope of a protein of a related target and a second peptide to a second T-cell epitope of the same related target. Herein, “multivalent vaccine” refers to any vaccine where the immunogenic effect is directed to more than one antigen. It is understood that a multivalent vaccine can comprise multiple components which can be formulated in the same mixture, in separate mixtures administered simultaneously with the first antigen. Likewise, the disclosed methods can comprise the simultaneous or separate administration of multiple vaccines. Thus, it is contemplated herein is the administration of a second, third, fourth, or fifth antigen, wherein the second, third, fourth, or fifth antigen is administered in a separate vaccine administered at the same time as or 1, 2, 3, 4, 5, 6, 10, 14, 18, 21, 30, 60, 90, 120, or 180 days (or any number of days in between) after the first antigen. Moreover, it is contemplated that the antigens provided in the mixture can come from the same or different or unrelated targets. Thus, it is contemplated herein that the antigens can be the same antigen. It is also contemplated herein that the antigens are related to heterologous antigens. For example, disclosed are methods of producing memory T-cells or protection comprising administering to a subject a mixture comprising a first antigen related to a first target and a second antigen related to a second target. Therefore, specifically contemplated are mixtures comprising, for example, a gag protein from HIV and the LLO₉₁₋₉₉ peptide from L. Monocytogenes.

Herein “subject” refers to the recipient of the mixture. It is understood and herein contemplated that “subject” can refer to a mammal, human, mouse, rat, guinea pig, monkey, chimpanzee, dog, cat, pig, cow, horse, or chicken.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed peptides, such as SEQ ID NO:1, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant Ga. (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

The methods and compositions disclosed herein provide accelerated and increased target-specific T-cell immunity. It is understood that one application of these methods is in the production and manufacture of vaccines. Thus, specifically disclosed and herein contemplated are methods of making a vaccine to an antigen comprising mixing a dendritic cell with the antigen and administering the mixture to a subject, wherein the mixture increases the number of memory T-cells specific to the antigen in the subject. It is understood that one of the benefits of the disclosed methods is accelerated production of target-specific T-cells. The accelerated production of target-specific T-cells occurs for both memory and effector T-cells. Therefore, specific disclosed are methods of making a vaccine to an antigen comprising mixing dendritic cells with the antigen and administering the mixture to a subject, wherein the mixture accelerates the production of memory T-cells specific to the antigen in the subject. Thus, disclosed herein are methods of accelerating the production of a protective amount of central memory T-cells in a subject to an antigen comprising mixing dendritic cells with the antigen and administering the mixture to the subject. Also disclosed are methods of accelerating the production of a protective amount of central memory T-cells in a subject to multiple antigens comprising mixing dendritic cells with the antigens and administering the mixture to the subject. Also disclosed are methods of making a vaccine, wherein the mixture also accelerates the production of the number of effector cells. Thus, disclosed herein are methods of making a vaccine to an antigen comprising mixing dendritic cells with the antigen and administering the mixture to a subject, wherein the mixture accelerates the production of the effector and memory T-cells specific to the antigen in the subject.

The methods disclosed herein can also be used to accelerate the transition from effector T cell to memory T cell. Accelerating the rate of this transition can lead to the establishment of memory earlier than would occur absent the disclosed methods. This has the advantage of conferring immunological protection against an antigen in a subject earlier than would otherwise be possible. Thus, disclosed herein are methods of making a vaccine to an antigen comprising mixing dendritic cells with the antigen and administering the mixture to a subject, wherein the mixture accelerates the transition from effector to memory T-cells specific to the antigen in the subject.

### C. METHODS OF USING THE COMPOSITIONS

1. Method of Treating Cancer

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T-cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### D. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular dendritic cell-peptide combination, such as DC-LLO₉₁₋₉₉ is disclosed and discussed and a number of modifications that can be made to a number of molecules including the DC-LLO₉₁₋₉₉ are discussed, specifically contemplated is each and every combination and permutation of DC-LLO₉₁₋₉₉ and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Nucleic Acids

There are a variety of molecules disclosed herein that are nucleic acid based, including for example the nucleic acids that encode, for example, the Listeria monocytogenes epitope LLO₉₁₋₉₉ as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an intemucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

2. Peptides

a) Protein Variants

As discussed herein there are numerous variants of the proteins that are known and herein contemplated. In addition, to the known functional strain variants there are derivatives of the proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinan T-cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinanT-cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | Ala; A |
| allosoleucine | AIle |
| arginine | Arg; R |
| asparagine | Asn; N |
| aspartic acid | Asp; D |
| cysteine | Cys; C |
| glutamic acid | Glu; E |
| glutamine | Gln; Q |
| glycine | Gly; G |
| histidine | His; H |
| isolelucine | Ile; I |
| leucine | Leu; L |
| lysine | Lys; K |
| phenylalanine | Phe; F |
| proline | Pro;P |
| pyroglutamic | Glu |
| acidp | |
| serine | Ser; S |
| threonine | Thr; T |
| tyrosine | Tyr; Y |
| tryptophan | Trp; W |
| valine | Val; V |

TABLE 2
Amino Acid Substitutions
Original Residue Exemplary
Conservative Substitutions,
others are known in the art.

Ala; ser
Arg; lys; gln
Asn; gln; his
Asp; glu
Cys; ser
Gln; asn, lys
Glu; asp
Gly; pro
His; asn; gln
Ile; leu; val
Leu; ile; val
Lys; arg; gln;
Met; Leu; ile
Phe; met; leu; tyr
Ser; thr
Thr; ser
Trp; tyr
Tyr; trp; phe
Val; ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant hosT-cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂ —, —CH═CH— (cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as a vaccine, for treating, inhibiting, or preventing an HIV infection, for example, the efficacy of the therapeutic antibody can be assessed in various ways well known to the skilled practitioner. For instance, one of ordinary skill in the art will understand that a composition, such as a vaccine, disclosed herein is efficacious in treating or inhibiting an HIV infection in a subject by observing that the composition reduces viral load or prevents a further increase in viral load. Viral loads can be measured by methods that are known in the art, for example, using polymerase chain reaction assays to detect the presence of HIV nucleic acid or antibody assays to detect the presence of HIV protein in a sample (e.g., but not limited to, blood) from a subject or patient, or by measuring the level of circulating anti-HIV antibody levels in the patient. Efficacy of the administration of the disclosed composition may also be determined by measuring the number of CD4⁺ T-cells in the HIV-infected subject. An antibody treatment that inhibits an initial or further decrease in CD4⁺ T-cells in an HIV-positive subject or patient, or that results in an increase in the number of CD4⁺ T-cells in the HIV-positive subject, is an efficacious antibody treatment.

The disclosed compositions can be administered prophylactically. For example, vaccines that inhibit HIV disclosed herein may be administered prophylactically to patients or subjects who are at risk for HIV, being exposed to HIV or who have been newly exposed to HIV. In subjects who have been newly exposed to HIV but who have not yet displayed the presence of the virus (as measured by PCR or other assays for detecting the virus) in blood or other body fluid, efficacious treatment with an antibody partially or completely inhibits the appearance of the virus in the blood or other body fluid.

4. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Accelerated CD8⁺ T-Cell Memory After Dendritic Cell Vaccination

a) Results and Discussion

Memory CD8⁺ T-cells are critical for resistance to many infections; however, generation of sufficient numbers of these cells by vaccination is often difficult (S. M. Kaech, et al. (2002) Nat. Rev. Immunol. 2, 251-62; J. Sprent and C. D. Surh (2002) Annu. Rev. Immunol. 20, 551-579), a limitation that can be overcome by booster immunizations to increase memory cell numbers (D. L. Woodland (2004) Trends Immunol. 25, 98-104). Efficient amplification of memory cell numbers generally requires several months between priming and booster immunizations, to allow the initially stimulated antigen-(Ag)-specificCD8⁺ T-cells to differentiate into memory T-cells with the capacity to undergo vigorous secondary expansion S. M. Kaech, et al. (2002) Cell 111, 837-851; E. J. Wherry et al., (2003) Nat. Immunol. 4, 225-234). The factors that regulate the delayed appearance of Ag-specific CD8⁺ T-cells with memory characteristics after infection or vaccination are unknown, however, it is apparent that immunization strategies that permit early boosting of memory cell numbers would improve vaccine efficacy.

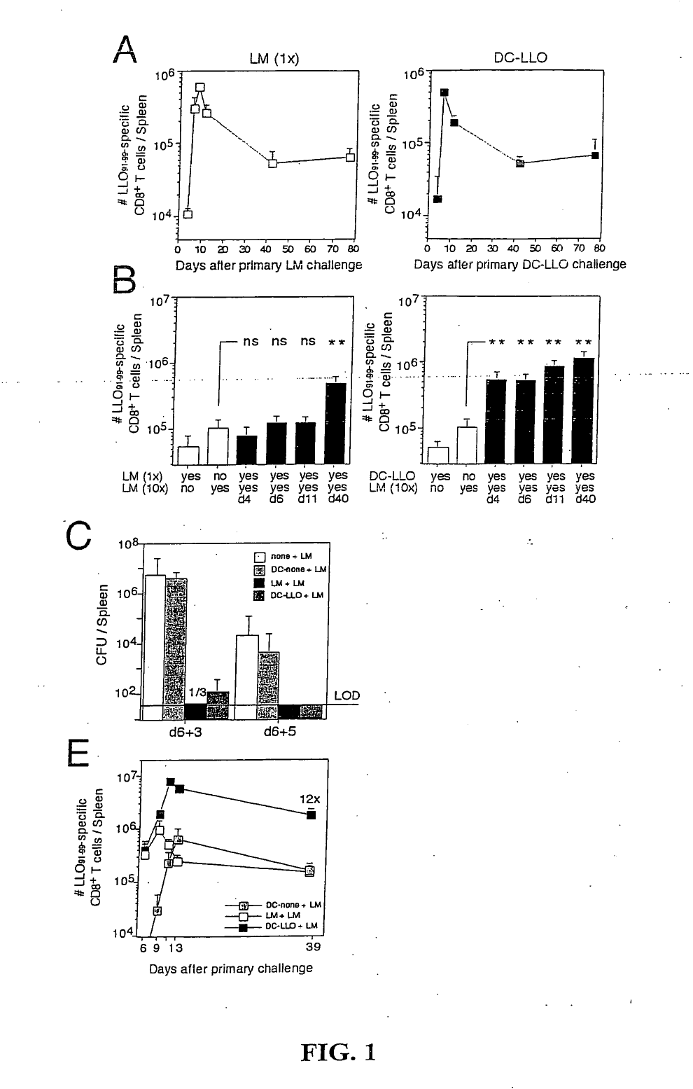

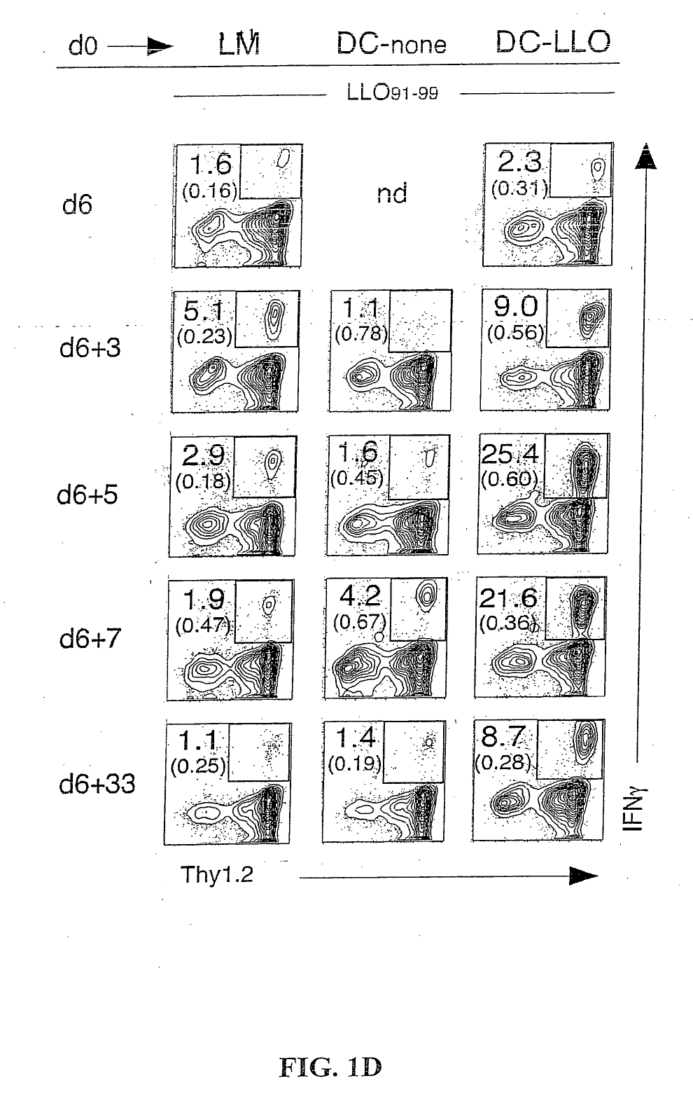

After infection with Listeria monocytogenes (LM, 0.1 LD₅₀) or immunization with LLO₉₁₋₉₉ (SEQ ID NO: 1) peptide-coated DC (DC-LLO), BALB/c mice had similar numbers of LLO₉₁₋₉₉-specific CD8⁺ T-cells during the expansion, contraction and memory phases of the immune response (FIG. 1A). Booster infection with 1.0 LD₅₀ of LM at day 40 after initial LM infection (LM+LM) resulted in secondary memory cell numbers 40 days later that were 5-10-fold increased compared to mice that received only the first or second infection (FIG. 1B). However, as previously observed (P. Wong, et al. (2004) J. Immunol. 172, 7239-45), the same dose of booster LM infection at days 4, 6 or 11 after initial LM infection failed to increase memory CD8⁺ T-cell numbers compared to mice that received only the booster infection (FIG. 1B). In marked contrast, booster infection as early as 4, 6, or 11 days after initial peptide-DC immunization (DC+LM) resulted in significantly higher (6-10-fold, p<0.05) memory cell numbers than achieved with the booster infection alone (FIG. 1B). Thus, peptide-DC immunization dramatically shortened the interval required for amplification of Ag-specific memory CD8⁺ T-cell numbers by booster infection.

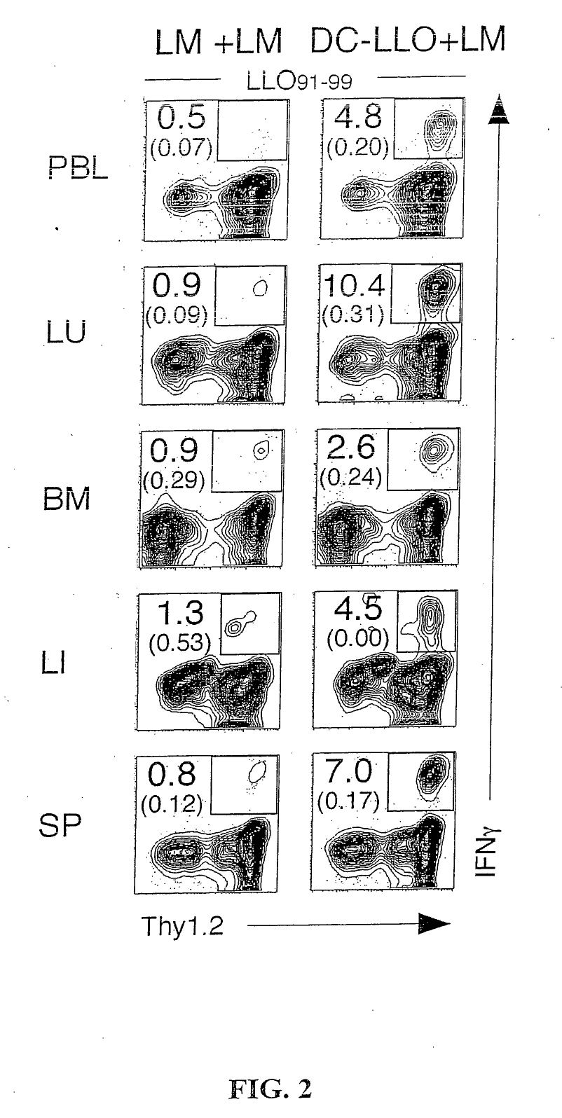

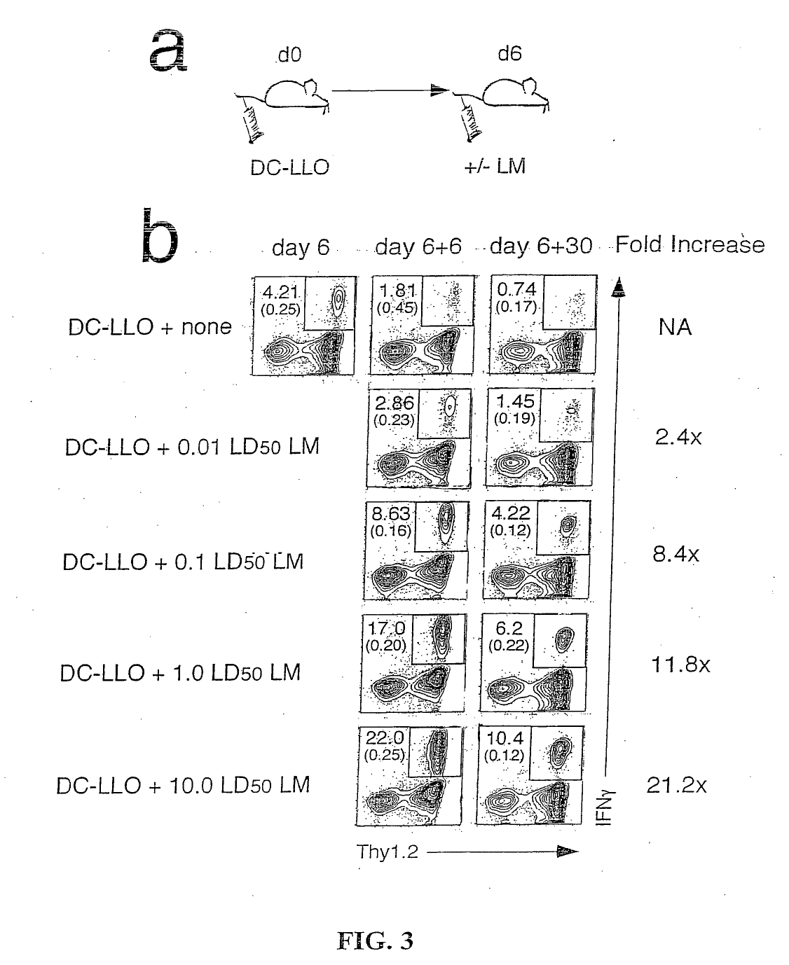

After booster infection at day 6, LM-infected and peptide-DC immunized mice rapidly cleared the infection, whereas mice immunized with uncoated DC and naïve mice did not control the infection (FIG. 1C). Therefore, the increased secondary memory numbers in DC+LM mice are not a result of delayed clearance of the booster infection compared to LM+LM mice. To determine the pathway resulting in elevated memory cell numbers, the number of LLO₉₁₋₉₉-specific CD8⁺ T-cells was determined at day 6 after initial immunization and at various days after booster infection in LM+LM and DC (±peptide)/LM mice (FIGS. 1D and E). Ag-specific CD8⁺T-cells in LM+LM mice increased ˜2-3-fold between day 6 and 9 and then underwent a pronounced contraction to stable memory cell numbers that were not elevated compared to mice receiving a single infection with LM (FIG. 1e). In contrast, Ag-specific CD8⁺ T-cells in DC+LM mice underwent substantial secondary expansion, reaching peak numbers at day 11 that were 25-fold higher than found at day 6. Ag-specific CD8⁺ T-cells in DC+LM mice then contracted; however, the resulting memory cell number was 12-fold higher than in LM+LM mice, and the elevated memory cell numbers were stable for at least 100 days. Immunization with uncoated DC did not lead to elevated memory levels after LM-booster infection; indicating that amplification of memory cell numbers in DC+LM mice did not result from either increased numbers of DC or non-specific alterations in the host caused by injection of DC. In addition, elevated numbers of memory T-cells in DC+LM immunized mice did not result from trapping of these cells in the spleen as they were observed in all tissues analyzed (FIG. 2). Finally, it was observed that increased memory cell numbers in DC+LM mice boosted with as few as 100 LM (0.01 LD₅₀) increased memory T-cell numbers, while boosting with 100,000 LM elevated memory T-cell numbers by >20-fold (FIG. 3). Thus, in contrast to effector cells generated by LM-infection, Ag-specific CD8⁺ T-cells at day 6 after peptide-DC immunization are capable of substantial secondary expansion and trafficking to tissues in response to even very weak booster infection.

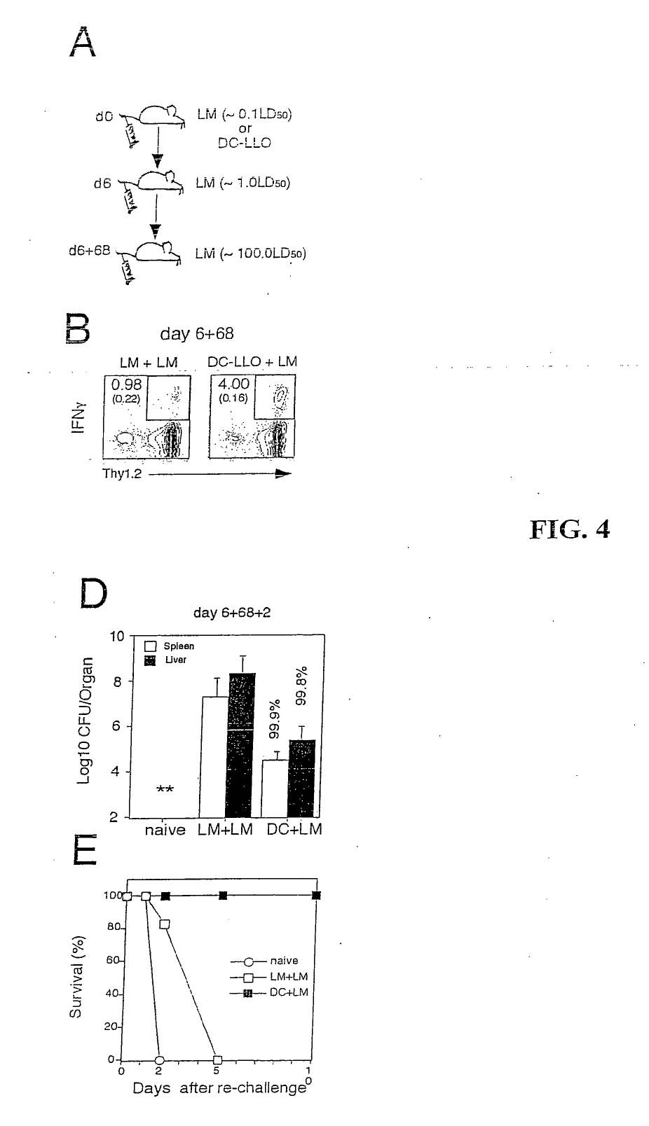

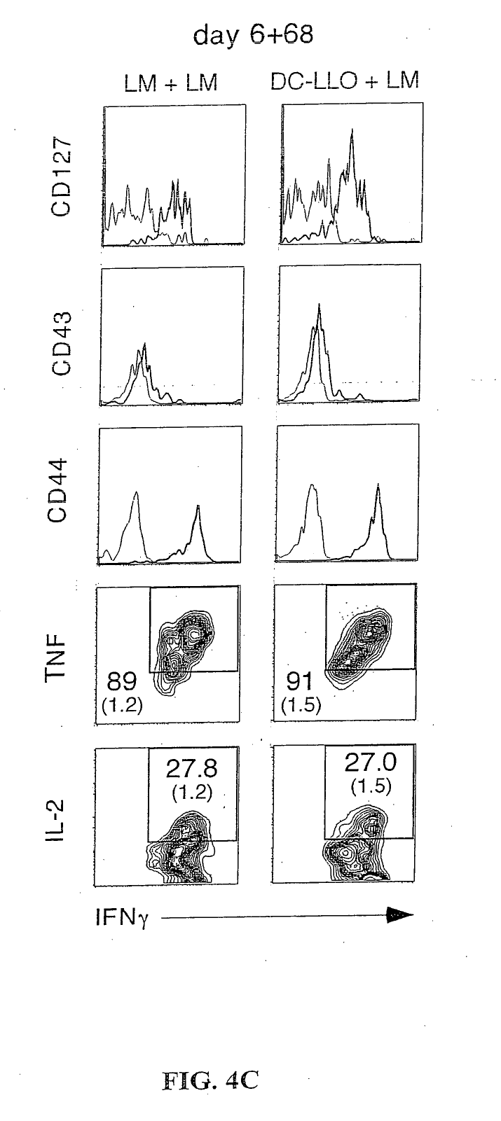

In a separate experiment (FIG. 4A), DC+LM mice contained ˜4-5-fold more LLO₉₁₋₉₉-specific CD8⁺ T-cells than LM+LM mice at day 68 after booster infection (FIG. 4B), which allowed these mice to control and survive high dose (100 LD₅₀) LM-infection that was lethal in naïve and LM+LM mice. However, Ag-specific CD8⁺ T-cells from both groups of mice exhibited a memory phenotype (CD44^{hi}, IL-7R⁺(K. S. Schluns, et al. (2000) Nat. Immunol. 1, 426-32 ; K. S. Schluns and L. Lefrancois (2003) Nat. Rev. Immunol. 3, 269-279; S. M. Kaech et al. (2003) Nat. Immunol. 4, 1191-1198), low levels of the CD43 isoform detected by the 1B11 mAb (L. E. Harrington, et al. (2000) J. Exp. Med. 191, 1241-1246) and the same fraction of cells producing both IFN-65 and TNF or IFN-65 and IL-2 after Ag-stimulation (E. J. Wherry, et al. (2003) J. Virol. 77, 4911-27). To assess the impact of increased memory cell numbers on immune protection, DC+LM and LM+LM mice and naïve controls were challenged with a 100 LD₅₀ of LM and measured bacterial numbers and survival. At day 2 after challenge, all naïve mice had died, LM+LM immunized mice had ˜10⁸ bacteria in their organs, whereas DC+LM immunized mice had 1000-fold fewer bacteria in the same organs (FIG. 4D). The reduced number of bacteria in DC+LM mice correlated with 100% survival whereas the LM+LM mice all died by day 5 after challenge infection (FIG. 4E). Therefore, the Ag-specific T-cells at d68 in DC+LM mice exhibit a memory phenotype and their increased number conferred a substantial survival advantage in response to high-dose infection.

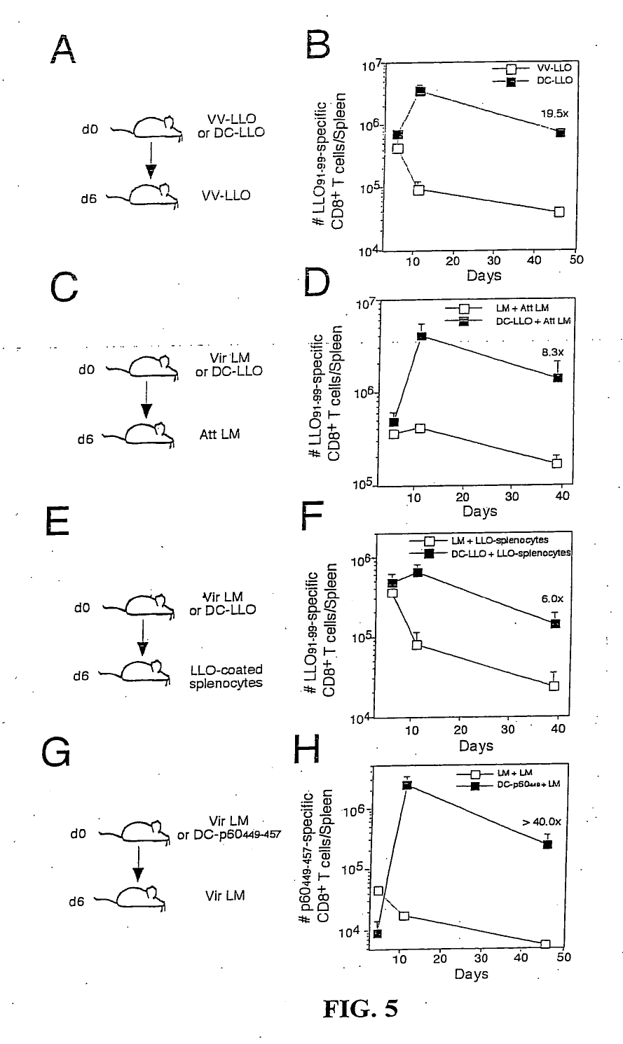

It is unlikely that booster immunizations in humans would be given with an agent such as virulent LM. Thus, the response of peptide-DC immunized mice to booster immunizations representing a range of virulence (FIGS. 5A, C and E) was evaluated. Boosting of peptide-DC vaccinated mice at day 6 with recombinant vaccinia virus expressing the LLO₉₁₋₉₉ epitope (L. L. An, et al. (1996) Infect. Immun. 64, 1685-1693) (FIG. 5A, 5B), attenuated actA-deficient LM (R. A. Brundage, et al. (1993) Proc. Nat. Acad. Sci. U. S. A. 90, 11890-4) (FIG. 5C, 5D), and even LLO₉₁₋₉₉-coated syngeneic spleen cells (FIG. 5E, 5F) all resulted in substantially elevated memory numbers 40 days later compared to infected mice given the same booster immunization. The ability of LLO₉₁₋₉₉-coated spleen cells to boost at d6 after DC-immunization but not LM-infection (FIG. 5G, 5F) indicates that antibody (Mackaness, G. B. (1962) J. Exp. Med. 1, 381-406; Edelson, B. T., et al. (1999) J. Immunol. 163, 4087-409) or other concurrent T-cell responses do not prevent the secondary response in LM-infected mice. Together, the results indicate a fundamental difference in the developmental stage of Ag-specific T-cells at d6 after DC-immunization compared to infection. In addition, these data demonstrate that the accelerated secondary response of CD8⁺ T-cell in DC-immunized mice can be stimulated by infectious and non-infectious booster immunizations.

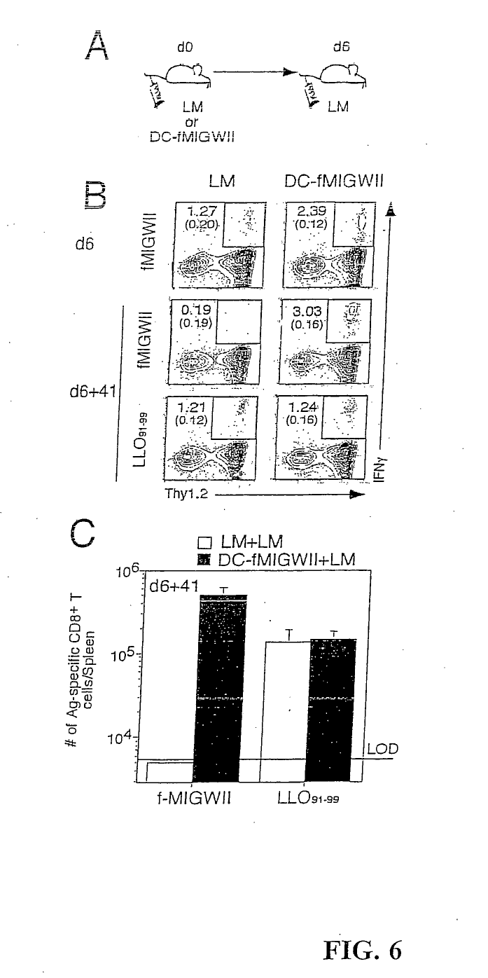

Many vaccines stimulate weak CD8⁺ T-cell memory, and prime-boost regimens are required to generate sufficient memory T-cells to achieve protective immunity (D. L. Woodland (2004) Trends Immunol. 25, 98-104). To determine if peptide-DC immunization would permit an accelerated amplification of memory cell numbers against weak antigens, mice were vaccinated with DC-coated with p60₄₄₉₋₄₅₇ (SEQ ID NO: 2), a subdominant LM antigen (D. H. Busch, et al. (1998) Immunity 8, 353-62), and boosted 6 days later with LM (FIG. 5G). Peptide-DC immunized mice had <10⁴ p60₄₄₉₋₄₅₇-specific CD8⁺ T-cells/spleen at day 6 compared to ˜5×10⁴ p60₄₄₉₋₄₅₇-specific CD8⁺ T-cell in LM infected mice (FIG. 5H). Booster LM infection did not generate high numbers of p60₄₄₉₋₄₅₇-specific CD8⁺ T-cell in mice that initially received LM, these cells were at or below the level of detection at day 40 after the boost. In contrast, the number of p60₄₄₉₋₄₅₇-specific CD8⁺ T-cells in peptide-DC immunized mice increased by ˜300-fold by day 5 after booster infection and these mice generated >40-fold higher memory cell numbers than achieved in LM+LM mice. Amplification of weak memory cell responses was not limited to MHC class Ia-restricted epitopes, but also occurred after vaccination with DC-coated with the H2-M3 (class Ib) restricted f-MIGWII (SEQ ID NO: 3) epitope from LM (K. M. Kerksiek, et al. (1999) J. Exp. Med. 190, 195-204; S. E. Hamilton, et al. (2004) Nat. Immunol. 5, 159-168) (FIG. 6). Thus, peptide-DC immunization can be used to rapidly amplify memory cell numbers even in response to weak antigens.

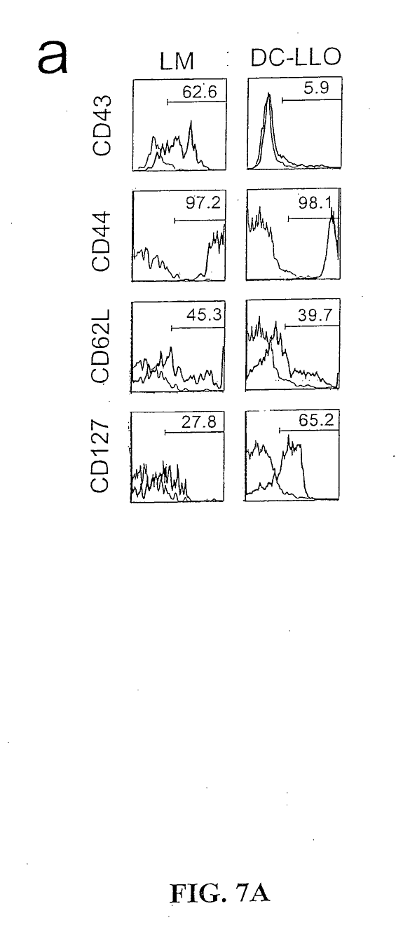

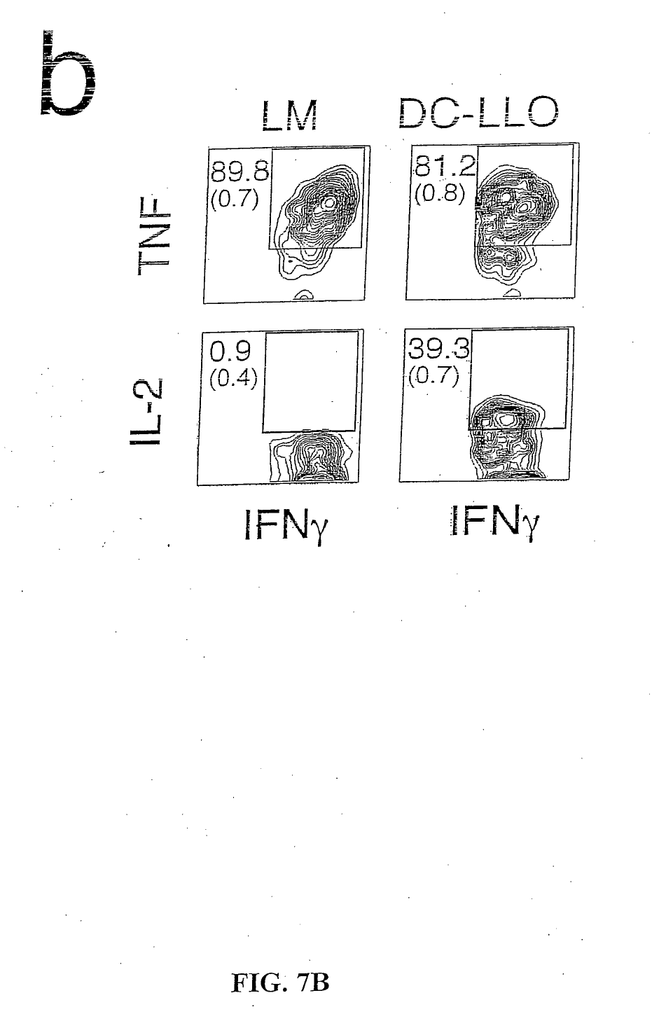

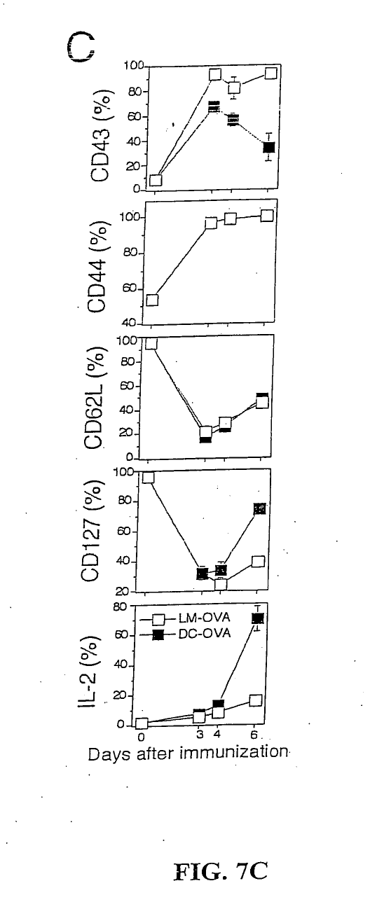

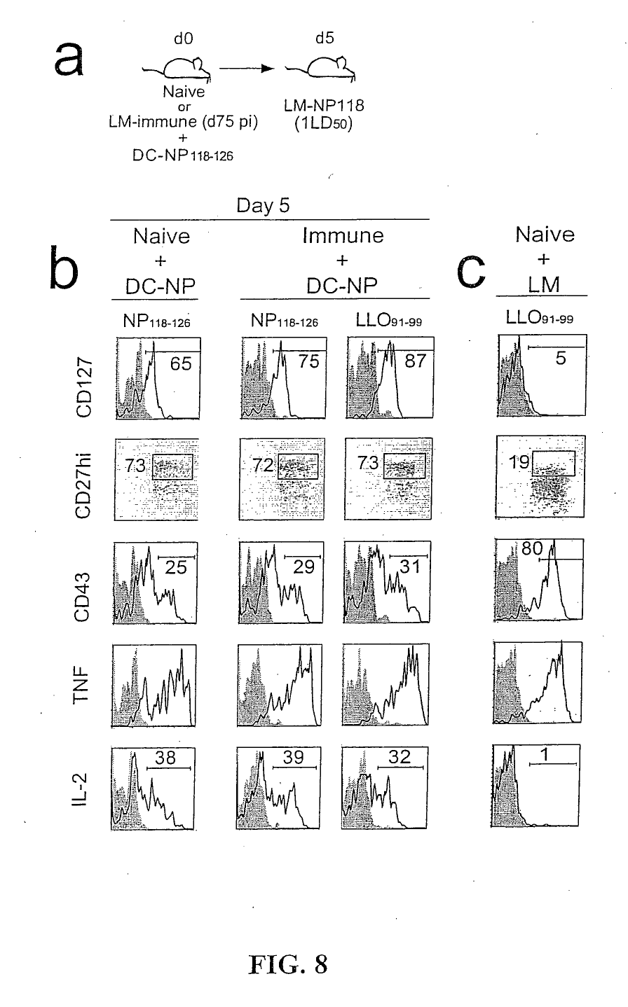

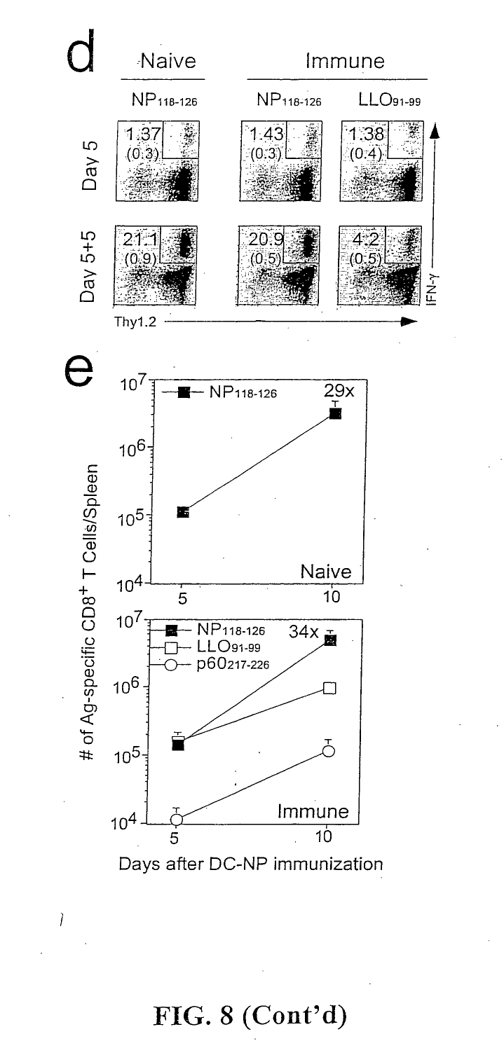

Effector and memory CD8⁺ T-cells exhibit both phenotypic and functional differences (S. M. Kaech, et al. (2002) Nat. Rev. Immunol. 2, 251-62; R. A. Seder and R. Ahmed (2003) Nat. Immunol. 4, 835-42). The majority of Ag-specific CD8⁺ T-cells at day 6 after LM-infection were CD44^{hi}, CD127^{lo}, CD43(1B11)^{hi}, failed to produce IL-2 after Ag-stimulation (FIGS. 7A and 7B) and therefore expressed an effector cell phenotype (S. M. Kaech, et al. (2002) Nat. Rev. Immunol. 2, 251-62; R. A. Seder and R. Ahmed (2003) Nat. Immunol. 4, 835-42). In striking contrast, Ag-specific CD8⁺ T-cells at day 6 after peptide-DC immunization were CD44^{hi}, CD127^{hi}, CD43(1B11)^{lo}, (FIG. 7A) and ˜40% produced IL-2 after Ag-stimulation (FIG. 7B), these cells exhibited characteristics of memory T-cells found 40 or more days after infection (FIG. 4B and (S. M. Kaech, et al. (2002) Cell 111, 837-851). To confirm that early DC-stimulated T cells possess a memory phenotype, LM-immune mice (containing memory LM-specific T cells) were vaccinated with LCMV NP₁₁₈-126-coated-DC and determined the phenotype of d75 LM-stimulated memory cells and d5 DC-stimulated T-cells (DC-NP), in the same immune mice. Both populations display similar memory phenotype (CD44^{hi}, CD127^{hi}, CD43 (1B11)^{lo} and >30% produced IL-2 after Ag-stimulation), including high levels of CD27 expression, another marker of functional memory cells (Hendriks, J., et al. (2000) Nat. Immunol. 1, 433-440) (FIG. 8A, 8B). Again, the phenotype of DC-stimulated cells differed dramatically from LM-stimulated effector cells (FIG. 8C). It was also noted that DC-stimulated CD8⁺ T-cells undergo normal contraction beginning at d7 (FIG. 1A) even though the majority of the cells express the IL-7R at d6 (FIG. 8B). These data were not expected in light of recent results suggesting that T-cells expressing high levels of the IL-7R survive contraction (Kaech, S. M. et al. (2003) Nat. Immunol. 4, 1191-1198; V. P. Badovinac, et al. (2004) Nat. Immunol. 5, 809-817; Huster, K. M. et al. (2004) Proc. Natl. Acad. Sci. U.S.A. 101, 5610-5615).

Finally, the d5 DC-NP stimulated CD8⁺ T-cells in both naive and LM immune mice were able to undergo vigorous secondary expansion after booster infection with LM expressing the NP₁₁₈-126 epitope (Shen, H., et al. (1998) Cell 92, 535-545). In fact, secondary expansion was similar in DC-NP immunized naïve (29-fold) and LM-immune (34-fold) mice (FIG. 8D, 8E) despite the presence of other LM specific CD8⁺ T-cells in immune mice that also underwent secondary expansion (FIG. 8D, 8E). Thus, DC-stimulated early memory cells can be boosted even in the presence of pre-existing immunity. Together, these data show that DC-immunization generates Ag-specific CD8⁺ T-cells with both the phenotype and function of true memory cells within 5-6 days after immunization.

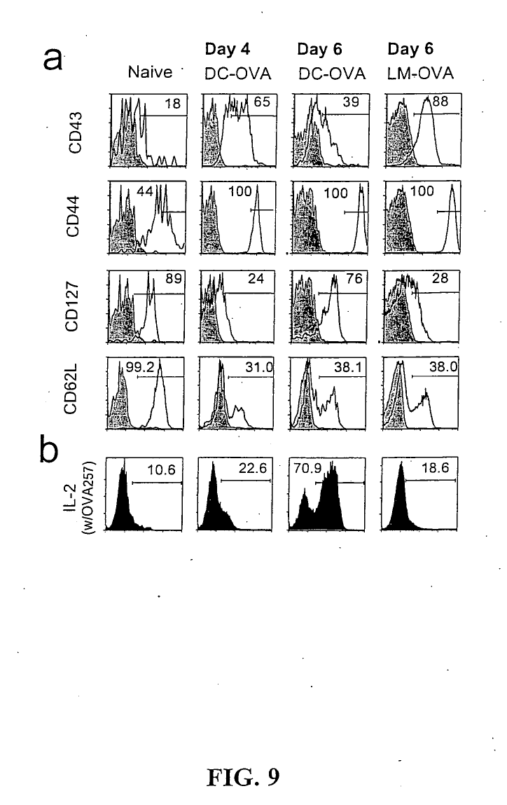

The data indicate that DC immunization either directly generated or accelerated the generation of Ag-specific CD8⁺ T-cells with the phenotype and function of memory T-cells. To resolve these possibilities, sufficient numbers of naive Thy1.1 congenic OT-1 TCR-Tg CD8⁺ T-cells (specific for amino acids 257-254 of ovalbumin (OVA; SEQ ID NO: 6))(K. A. Hogquist et al. (1994) Cell 76, 17-27) were transferred into naïve Thy1.2 hosts to allow detection and analysis at day 3 and subsequent days after infection with LM expressing ovalbumin (LM-OVA(K. E. Foulds et al. (2002) J. Immunol. 168, 1528-1532)) or immunization with DC coated with the OVA₂₅₇₋₂₆₄ peptide (DC-OVA). Importantly, the majority of OT-1 cells at day 6 after DC-OVA immunization exhibited a memory phenotype (CD44^{hi}, CD127^{hi}, CD43(1B11)^{lo}) and produced IL-2 whereas the OT-1 cells at day 6 after LM-OVA infection displayed an effector phenotye (CD44^{hi}, CD127^{lo}, CD43(1B₁₁)^{hi}) and failed to produce IL-2 (FIGS. 7C and 9). Thus, the TCR-Tg cells recapitulated the phenotype and functional properties displayed by endogenous populations of Ag-specific CD8⁺ T-cells at day 6 after stimulation by DC immunization or infection (FIGS. 7A and 7B). In contrast to the differences observed at day 6, the majority of OT-1 cells at day 3 after LM-OVA infection or DC-OVA immunization exhibited an effector phenotype (CD44^{hi}, CD127^{lo}, CD43(1B11)^{hi}) and failed to produce IL-2 (FIG. 7C). Thus, DC-peptide immunization did not directly generate memory phenotype CD8⁺ T-cells, but instead, dramatically accelerated the transition from an effector population into cells with memory phenotype and function. Interestingly, despite the high frequency of Ag-specific CD8⁺ T-cells expressing the IL-7R at day 6 (FIG. 7), these cells underwent normal contraction beginning at day 7 after DC immunization (FIG. 1A). These data were not expected in light of recent results indicating that T-cells expressing high levels of the IL-7R survive contraction (S. M. Kaech et al. (2003) Nat. Immunol. 4, 1191-1198; V. P. Badovinac, et al. (2004) Nat. Immunol. 5, 809-817).

Multiple DC subsets are thought to play specific roles in the immune response (V. P. Badovinac, et al. (2004) Nat. Immunol. 5, 809-817; J. Banchereau et al. (2000) Annu. Rev. Immunol. 18; R. M. Steinman, et al. (2003) Annu. Rev. Immunol. 21, 685-711). Additionally, recent studies suggest that the CD8⁺ T-cell response program, including the contraction to memory levels (P. Wong and E. G. Pamer (2001) J. Immunol. 166, 5864-8; S. M. Kaech and R. Ahmed (2001) Nat. Immunol. 2, 415-22); M. J. van Stipdonk, et al. (2001) Nat. Immunol. 2, 423-9; V. P. Badovinac, et al. (2002) Nat. Immunol. 3, 619-26) is influenced by early inflammation after infection (V. P. Badovinac, et al. (2004) Nat. Immunol. 5, 809-817). Interestingly, preliminary analysis revealed rapid elevation of IL-1β, GM-CSF, IFN-γ, TNF, IL-6, IL-12, G-CSF, MIP-1α and RANTES protein in serum by 20 h after LM-infection; however, none of these inflammatory mediators were elevated in the serum of DC-immunized mice, indicating that accelerated generation of memory CD8⁺ T-cells may also result from a lack of infection-induced inflammatory signals during T-cell priming.

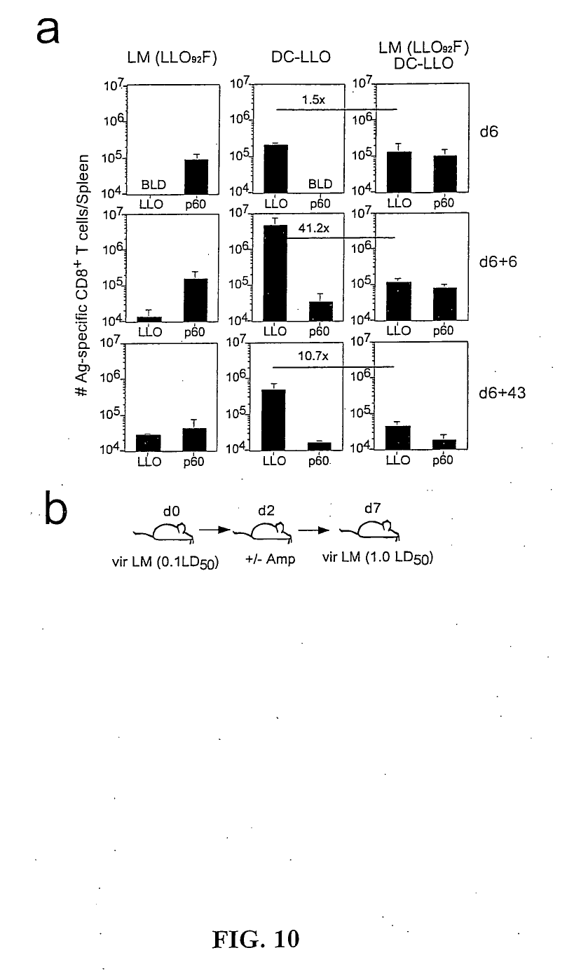

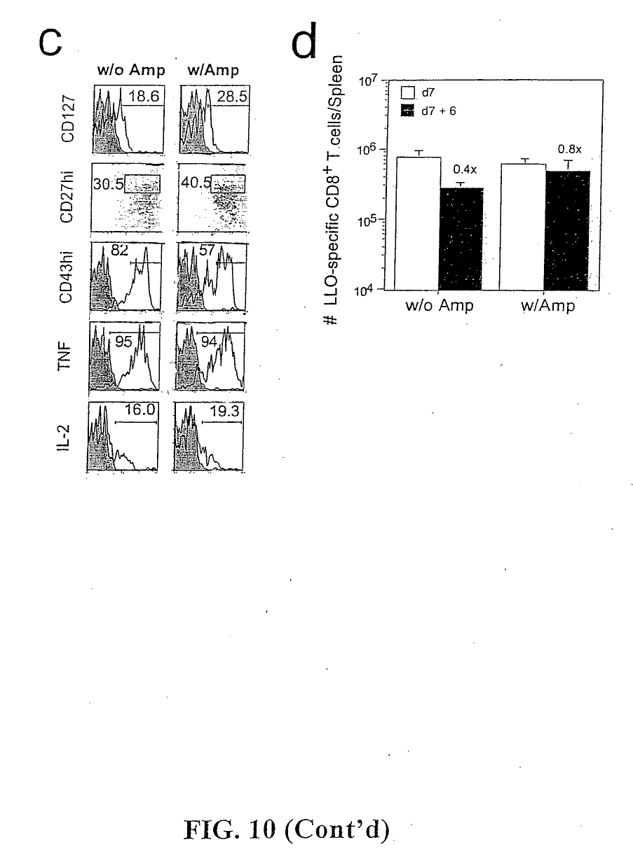

To distinguish between these possibilities, mice were injected with LLO₉₁₋₉₉-coated DC and/or a virulent LM carrying an epitope destroying mutation at residue 92 of LLO (SEQ ID NO: 5) and 6 days later, infected all groups with wild-type LM. This experimental design ensures that LLO₉₁₋₉₉-specific CD8⁺ T-cells are primed by the injected DC, in the presence or absence of LM infection. In contrast to the elevated memory numbers achieved in DC+LM mice, concurrent injection of peptide-coated DC and LM infection did not generate LLO₉₁₋₉₉-specific CD8⁺ T-cells able to undergo secondary expansion and generate higher memory levels after booster infection (FIG. 10A). These results indicate that the rate of memory cell generation was determined by whether priming occurred in the presence or absence of infection. Truncating LM-infection by antibiotic treatment at d2, which causes clearance of LM by d3 versus d6-7 in control mice (Mercado, R. et al. (2000) J. Immunol. 165, 6833-6839 (2000)), did not change the effector phenotype of Ag-specific T-cells at d7 or permit these cells to undergo secondary expansion after booster immunization (FIG. 10B). Thus, the duration of infection does not determine whether CD8⁺ T-cells can respond to early booster immunization. Again, these data suggest a fundamental difference in the Ag-specific CD8⁺ T-cells stimulated by DC-vaccination versus infection.

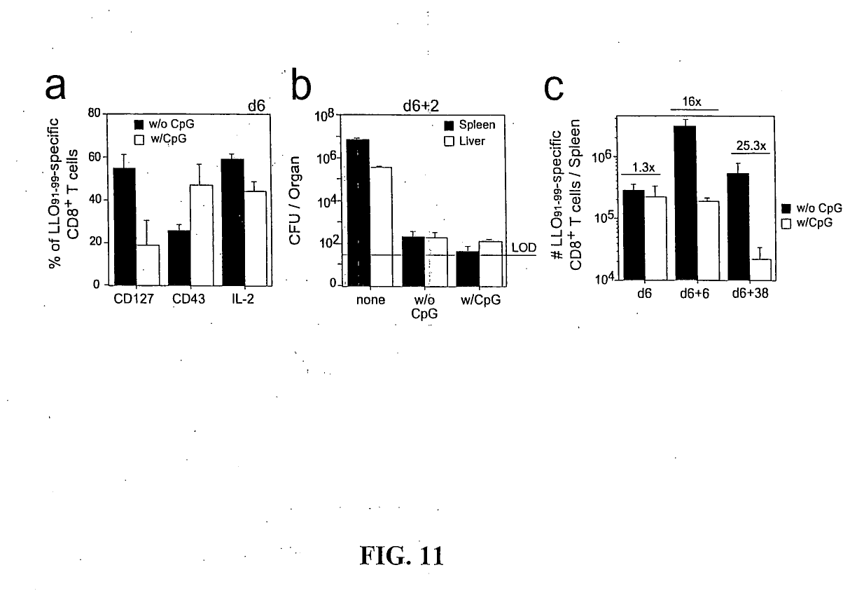

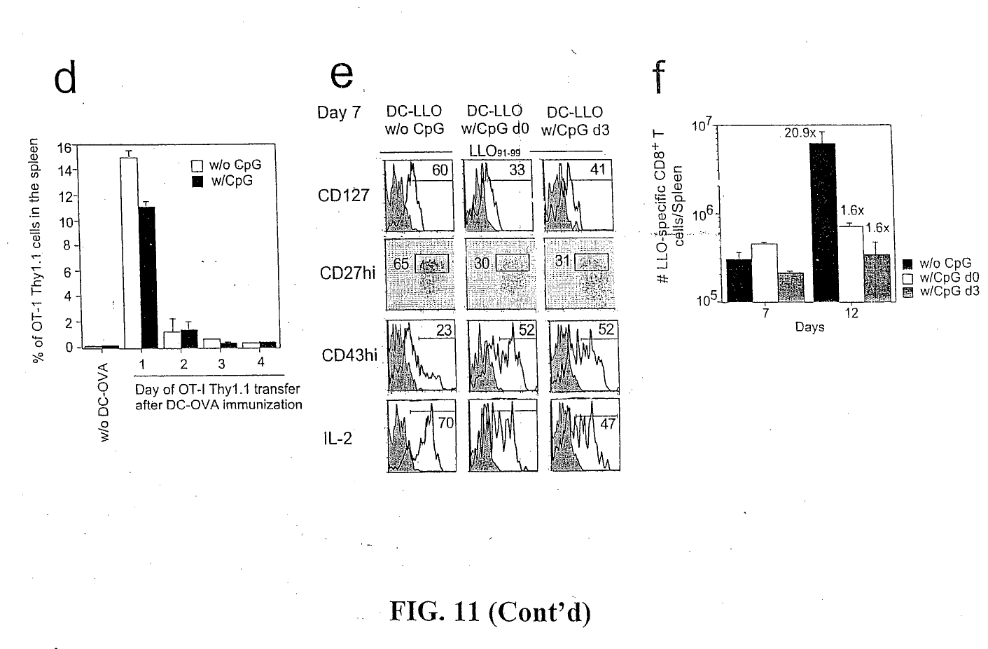

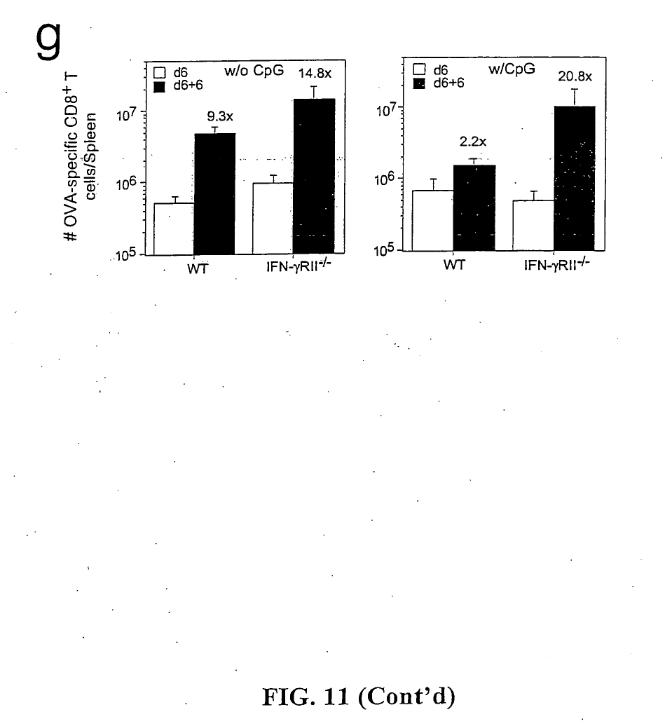

LM-infection results in substantial activation of the innate immune system, including the production of proinflammatory cytokines such as IL-12 and type I and type II IFN (Pamer, E. G. (2004) Nat. Rev. Immunol. 4, 812-823). To induce these same inflammatory mediators in the absence of infection, mice were immunized with LLO₉₁₋₉₉-coated-DC with or without CpG oligodeoxynucleotide 1826 (Krieg, A. M. (2003) Nat. Med. 9, 831-835 (2003); (Takeda, K., et al. (2003) Ann. Rev. Immunol. 21, 335-376). The CpG-treatment did not alter the magnitude of the LLO₉₁₋₉₉-specific CD8⁺ T-cell response at d6 after DC-immunization (FIG. 11A) or the ability of mice to clear the booster LM-infection (FIG. 11B). However, CpG-treatment substantially decreased the fraction of Ag-specific CD8⁺ T-cells with memory phenotype (CD127^{hi}, CD43^{lo}, FIG. 11C). Although only a modest decrease in the percent of IL-2 producing T-cells occurred in CpG-treated mice, (FIG. 11C), these cells were unable to respond to booster immunization (FIG. 11A). Thus, CpG-treatment prevents accelerated generation of memory CD8⁺ T-cells and early prime-boost response.

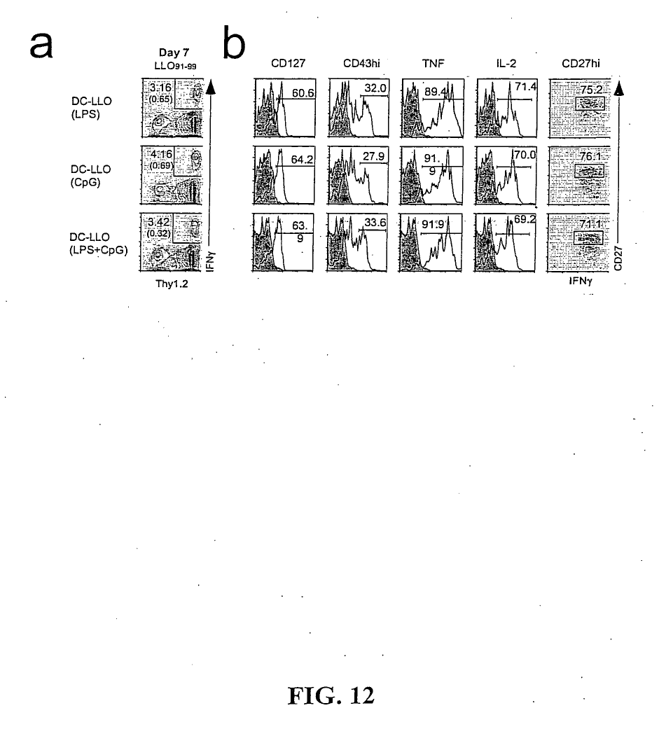

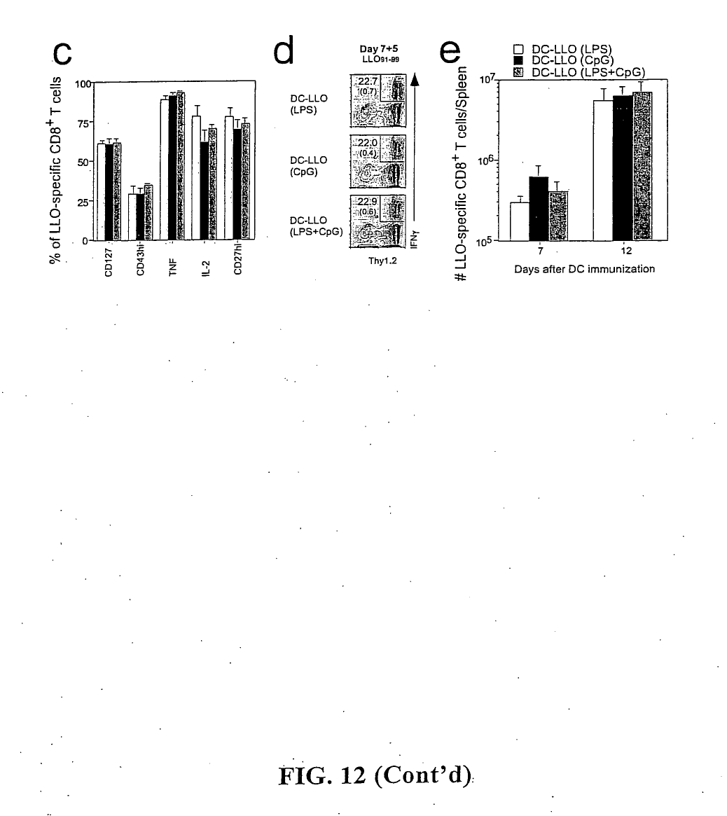

DC matured in vitro with LPS, CpG or LPS+CpG, all stimulated memory phenotype CD8⁺ T-cells at d6 after immunization, and these T-cells underwent vigorous secondary responses to LM booster immunization (FIG. 12). Thus, it is unlikely that CpG-treatment directly altered DC function to prevent early memory formation. CpG-treatment could alter the lifespan of the injected DC, and thus mimic infection by increasing the duration of antigen presentation. However, there was no difference in DC recovery between control and CpG-treated mice at various days after immunization (DC-became undetectable at 48 h after injection) and CpG-treatment did not alter the duration of DC-Ag display, detected by proliferation of naïve OT-1 TCR-tg cells injected at various days after DC-OVA-immunization (FIG. 11D). Thus, CpG-treatment does not alter the lifespan of the injected DC or increase the duration of DC-mediated Ag-presentation in vivo.

It remained possible that the CpG-treatment was acting indirectly through the injected DC to prevent early memory generation of Ag-specific CD8⁺ T-cells. To address this, DC-immunized mice received no CpG, CpG at d0 or CpG at d3, a time point where the DC are no longer detectable and DC-mediated Ag-presentation is dramatically reduced (FIG. 11D). Interestingly, CpG-treatment as late as d3 after DC-immunization prevented the generation of CD8⁺ T-cells with early memory phenotype (FIG. 11E) and inhibited the response to booster immunization as well as CpG-treatment on d0 (FIG. 11F). These data show that CpG does not act through the injected DC to prevent generation of early memory CD8⁺ T-cells.

Since T-cells do not express TLR9 (Wagner, H. (2002) Curr. Opin. Microbiol. 5, 62-69), the results suggest that CpG-treatment prevents early memory generation by inducing inflammatory cytokines that act to alter the program of the responding T-cells. To determine if CpG-stimulated IFN-γ (Krieg, A. M. (2003) Nat. Med. 9, 831-835 (2003); (Takeda, K., et al. (2003) Ann. Rev. Immunol. 21, 335-376) plays a role in preventing early memory generation after DC-immunization, WT or IFN-65 receptor (R) II-deficient mice were vaccinated with DC-OVA, ±CpG-treatment, and boosted the mice with LM-OVA at d6. DC-immunization stimulated a similar expansion of Ag-specific T-cells in WT and IFN-γRII-deficient mice and these cells underwent vigorous secondary expansion in response to d6 booster immunization (FIG. 11G). CpG-treatment of WT mice prevented substantial secondary expansion in response to d6 booster immunization. In striking contrast, CpG-treatment did not prevent the booster response in DC-vaccinated IFN-γRII-deficient mice. These data provide indicate that early inflammation, specifically involving IFN-γ, acts through the IFN-γR on T-cells to prevent early generation of memory characteristics. Thus, inflammatory signals received by the T-cells during or shortly after priming determine the rate at which T-cells acquire memory characteristics.

In summary, DC-immunization creates a situation where the naïve T-cells receive TCR and co-stimulatory signals in the absence of infection-induced inflammatory signals. As a consequence, the lack of inflammatory signals to the T-cells during priming dramatically accelerates acquisition of memory characteristics, permitting a shorter interval between priming and booster immunizations. Consistent with this notion, CpG-treatment to induce inflammation at the time of or shortly after DC-immunization prevented the accelerated generation of memory cells and the rapid prime boost response, despite the fact that the magnitude of the CD8⁺ T-cell response was unaltered and the CpG-treatment did not alter the lifespan of the injected DC or the duration of DC-mediated Ag-presentation. In addition, the failure of CpG-treatment to inhibit early memory formation after WT DC-immunization of IFN-γRII-deficient mice, an experimental design where the T-cells but not DC lack the IFN-γRII, indicates that specific inflammatory mediators acting directly on the T-cells determine the rate of memory CD8⁺ T-cell generation.

A major constraint on the success of DC-based cancer immunotherapy is the requirement for rapid generation of high numbers of effector and memory CD8⁺ T-cells specific for tumor antigens. These antigens are often tissue-specific self-peptides, where tolerance-induced limitations in the available T-cell repertoire may restrict the magnitude of the primary response. The results show that DC-immunization allows the rapid amplification of effector CD8⁺ T-cells specific for weak, subdominant antigens. Thus, this strategy addresses major limitations of DC-vaccination for cancer immunotherapy. Importantly, the amplification of memory CD8⁺ T-cell numbers in peptide-DC immunized mice occurs in response to a variety of booster immunizations, including those (vaccinia virus, attenuated LM, Ag-coated cells) that can be readily applied to humans.

b) Materials and Methods

(1) Mice, Listeria monocytogenes, Vaccinia virus, CpG, Peptide-Coated Splenocytes.

BALB/c (Thy1.2⁺, H-2^{d} MHC) and C57B1/6 (Thy1.2⁺, H-2^{b}) mice were obtained from the National Cancer Institute (Frederick, Md.). OT-I Tg Thy1.1⁺ mice were previously described (K. A. Hogquist et al., (1994) Cell 76, 17-27). Pathogen-infected mice were housed in the appropriate biosafety conditions. All mice were used at 8-16 weeks of age. Virulent (10403s and DP-L2528 (LM LLO92F, non-functional LLO₉₁₋₉₉ epitope)(H. G. Bouwer, et al. (1996) Infect. Immun. 64, 3728-3735) and attenuated (DP-L1942 (which is actA-deficient)(R. A. Brundage, et al. (1993) Proc. Nat. Acad. Sci. U. S. A. 90, 11890-4) and act A-deficient LM-OVA (LM expressing ovalbumin)(K. E. Foulds et al., (2002) J. Immunol. 168, 1528-1532)) L. monocytogenes strains were grown, injected i.v. and colony forming units (CFU) per spleen and gram of liver were determined on various days after infection as described (J. T. Harty, and M. J. Bevan (1995) Immunity 3, 109-17). Vaccinia-virus expressing LLO (VV-LLO) was provided by J. Lindsay Whitton (Scripps) and was propagated and injected i.p. as described (L. L. An, et al. (1996) Infect. Immun. 64, 1685-1693). CpG ODN 1826 (V. P. Badovinac, et al. (2004) Nat. Immunol. 5, 809-817) was injected i.p. at the time of DC immunization at 60 mg/mouse. Naive BALB/c splenocytes were coated with LLO₉₁₋₉₉ peptide (1 mM) at 37 C. for an hour, washed three times and injected i.v. into mice at the concentration of 2×10⁷ cells/mouse.

(2) Antibodies, Peptides.

The following monoclonal antibodies with appropriate combination of fluorochromes were used: anti-IFN-65 (clone XMG1.2, eBioscience), anti-CD8 (53-6.7, Pharmingen), anti-Thy1.2 (53-2.1, Pharmingen), anti-TNF (MP6-XT22, eBioscience), anti-CD127 (A7R34, eBioscience), anti-CD43 (1B11, Pharmingen), anti-CD44 (Pgp-1, Pharmingen), anti-IL-2 (JES6-5H4, Pharmingen), anti-CD62L (MEL-14, eBioscience), anti-CD25 (PC61, eBioscience), and isotype controls IgG2a, IgG2b, and IgG1 (clones eBR2a, KLH/G2b-1-2, eBRG1, respectively, eBioscience). Synthetic peptides—which represented defined L. monocytogenes LLO₉₁₋₉₉, p60₂₁₇₋₂₂₅ (SEQ ID NO: 4), p60₄₄₉₋₄₅₇, f-MIGWII, and lymphocytic choriomeningitis virus (LCMV) derived NP₁₁₈₋₁₂₆ (SEQ ID NO: 7) as well as OVA₂₅₇₋₂₆₄ were previously described (K. A. Hogquist et al., (1994) Cell 76, 17-27; D. H. Busch, et al. (1998) Immunity 8, 353-62; K. M. Kerksiek, et al. (1999) J. Exp. Med. 190, 195-204).

(3) Adoptive Transfer of OT-I.

Purfied naïve OT-I Thy1.1 cells (4×10⁵/mouse) were transferred into naïve C57B1/6 Thy1.2 mice and one day later the recipient mice were immunized either with actA-deficient LM-OVA (4×10⁶) or DC coated with OVA₂₅₇₋₂₆₄ peptide (4×10⁵ CD11c⁺ cells).

(4) Bone Marrow-Derived Dendritic Cells.

Bone marrow-derived CD11c⁺ DCs were generated by 5-7 d of culture in GM-CSF and IL-4 as described (S. E. Hamilton, et al. (2004) Nat. Immunol. 5, 159-168). Lypopolysaccharide (1 mg/ml; Sigma) was then added for 1 d to induce maturation, and peptide (1 mM) was added to cultures 2 h before cells were collected and extensively washed before injection. The resulting cell populations consisted of 50-80% CD11c+ cells. These cells were also H-2L^{d+}, B7.1⁺, B7.2⁺, CD8a⁻, I-A^{d+} and CD11b⁺. Based on percentage of CD11c+ cells (determined before injection), 2.5×10⁵ mature peptide coated DCs were injected intravenously.

(5) Quantification of Antigen-Specific CD8+ T-Cell Response.

The magnitude of the epitope-specific CD8⁺ T-cell response was determined by peptidestimulated intracellular staining for IFN-γ, IFN-γ/TNF, IFN-γ/IL-2 as described (V. P. Badovinac and J. T. Harty (2000) J. Immunol. Methods 238, 107-117). The percentage of IFN-γ⁺CD8⁺ T-cells in unstimulated samples from each mouse was subtracted from the peptide-stimulated value to determine the percentage of antigen-specific CD8⁺ T-cells. The total number of epitope-specific CD8⁺ T-cells per spleen was calculated from the percentage of IFN-γ⁺CD8⁺ T-cells, the percentage of CD8⁺ T-cells in each sample and total number of cells per spleen. The same procedure was used for detection of Ag-specific CD8⁺ T-cells obtained from various organs as previously described (V. P. Badovinac, et al. (2003) Immunity 18, 463-74). LLO₉₁₋₉₉-specific CD8⁺ T-cells were also detected by phycoerytrin-conjugated tetramer complexes as described (V. P. Badovinac, et al. (2003) Immunity 18, 463-74).

### 2. Example 2

### Applications of DV Immunization/Early Boost to Mouse Tumor Immunotherapy Models

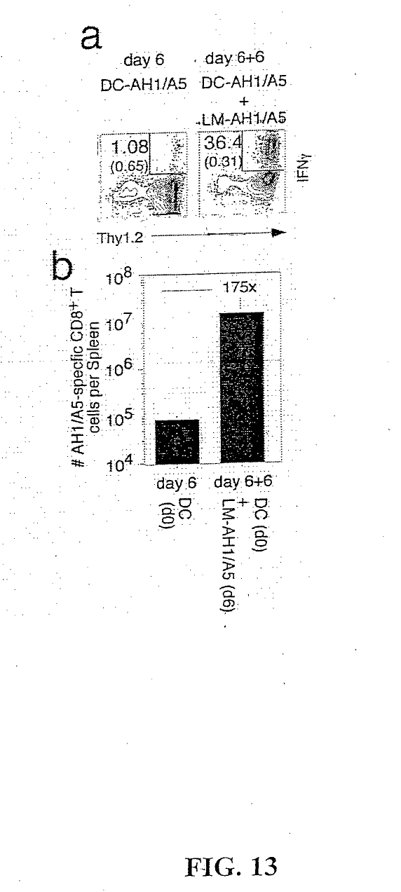

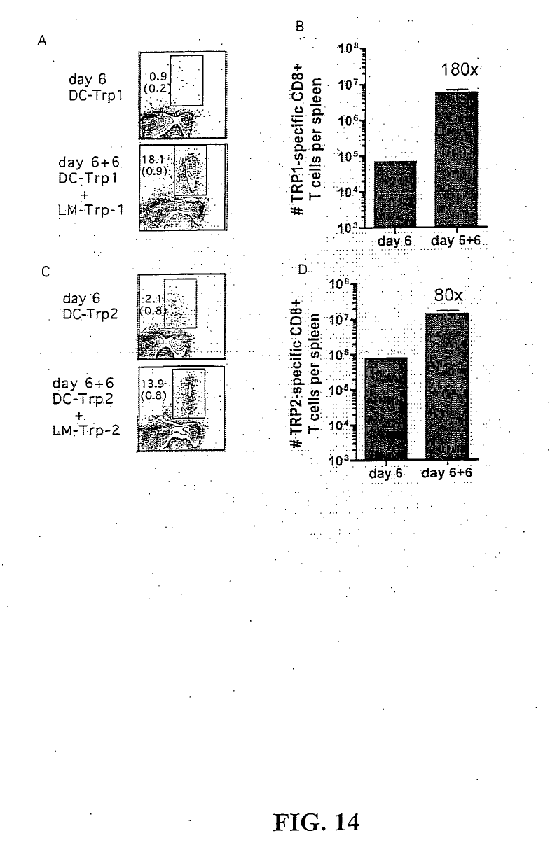

DC immunization and early booster immunization has the potential to rapidly generate large numbers of effector CD8 T cells and thus, overcome one of the limitations currently observed in the immunotherapy of malignancy. In support of this notion, DC immunization and early boosting was evaluated with a model, altered peptide epitope (AH1/AH5) (SEQ ID NO: 8) from the CT26 colon carcinoma model of BALB/c mice (FIG. 13) and also with a self peptides (Trp1 (SEQ ID NO: 9) and Trp2 (SEQ ID NO: 10), both from the tyrosinase protein) from the B16 melanoma model of C57B1/6 mice (FIG. 14). In both cases, initial immunization with DC followed by boosting with recombinant Listeria monocytogenes expressing the same antigen resulted in generation of extremely large effector CD8 T cell responses by d12-14 after immunization. In both cases, these effector populations gave rise to elevated numbers of functional memory CD8 T cells.

### 3. Example 3

### Application of DC Immunization/Early Booster to Enhance CD4 T Cell Mediated Resistance to leishmania

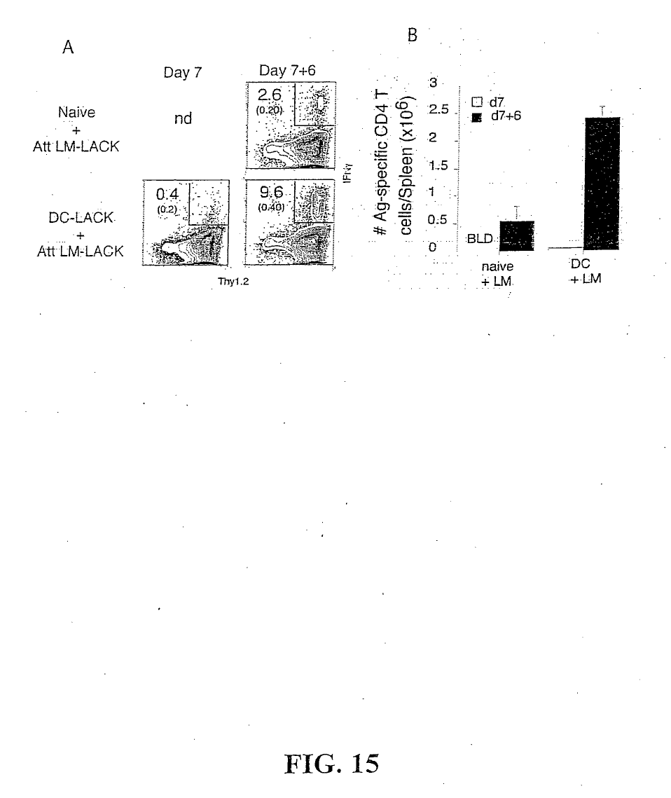

Protective immunity against the protozoan parasite Leishmania donovani, the cause of visceral leishmaniasis, is mediated by CD4 T cells. The DC immunization/early booster strategy was used to determine if the number of effector and memory CD4 T cells specific for the L. donnovani “LACK” peptide (SEQ ID NO: 11) could be amplified. As shown in FIG. 15, the number of effector and memory CD4 T cells was increased using the DC/early booster approach.

### 4. Example 4

### Application of DC Immunization/Early Booster to Enhance CD8 T Cell Mediated Resistance to Malaria

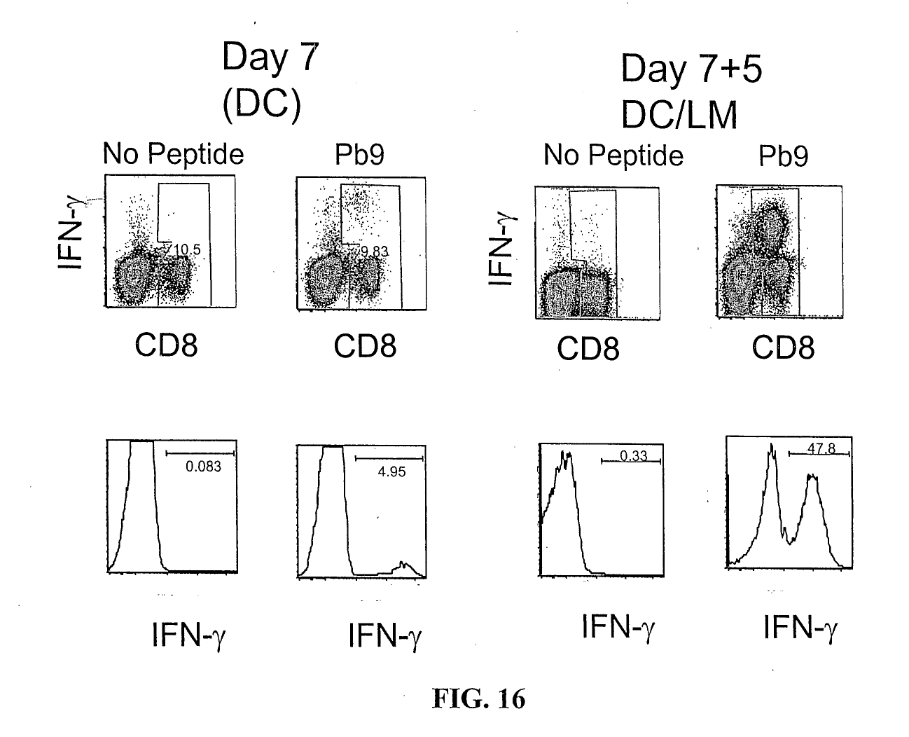

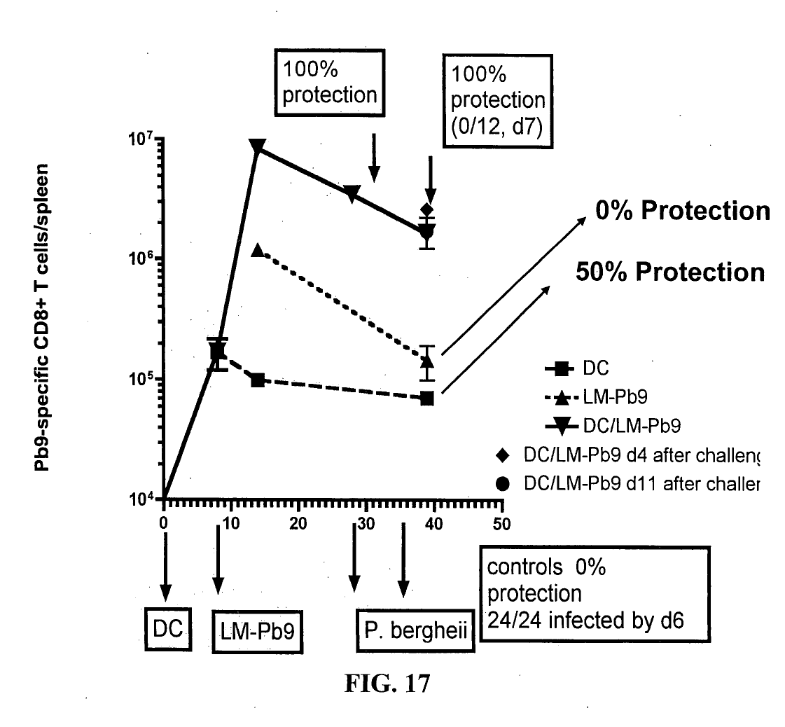

Generation of CD8 T cells able to recognize infected hepatocytes is a potentially important goal of vaccines against malaria parasites. However, most preclinical data on mouse models suggest that very large numbers of Ag-specific CD8 T cells are required to mediate sterilizing immunity to the malaria liver stage and that the protective effects of vaccination are relatively short lived. The DC immunization/early booster strategy was used to address these issues and protective immunity in a mouse model of malaria infection. Substantial CD8 T cell responses were generated against a defined malaria (Plasmodium bergheii) CD8 T cell epitope called Pb9 (SEQ ID NO: 12) using the DC-peptide/early boost approach (FIG. 16). Mice immunized in this way were completely protected from malaria challenge infection at 21 and 28 days after the boost (FIG. 17).

### F. REFERENCES

An, L. L., Pamer, E. & Whitton, J. L. A recombinant minigene vaccine containing a nonameric cytotoxic-T-lymphocyte epitope confers limited protection against Listeria monocytogenes infection. Infect. Immun. 64, 1685-1693 (1996).
Badovinac, V. P. & Harty, J. T. Intracellular staining for TNF and IFNgamma detects different frequencies of antigen-specific CD8(+) T-cells. J. Immunol. Methods 238, 107-117 (2000).
Badovinac, V. P. & Harty, J. T. Memory lanes. Nat. Immunol. 4, 212-213 (2003).
Badovinac, V. P., Hamilton, S. E. & Harty, J. T. Viral infection results in massive CD8+ T-cell expansion and mortality in vaccinated perforin deficient mice. Immunity 18, 463-474 (2003).
Badovinac, V. P., Messingham, K. A., Hamilton, S. E. & Harty, J. T. Regulation of CD8+ T-cells undergoing primary and secondary responses to infection in the same host. J. Immunol. 170, 4933-4942 (2003).
Badovinac, V. P., Porter, B. B. & Harty, J. T. CD8+ T-cell contraction is controlled by early inflammation. Nat. Immunol. 5, 809-817 (2004).
Badovinac, V. P., Porter, B. B. & Harty, J. T. Programmed contraction of CD8(+) T-cells after infection. Nat. Immunol. 3, 619-626. (2002).
Badovinac, V. P., Tvinnereim, A. R. & Harty, J. T. Regulation of antigen specific CD8(+) T-cell homeostasis by perforin and interferon-gamma. Science 290, 1354-1357 (2000).
Banchereau, J. et al. Immunobiology of dendritic cells. Ann. Rev. Immunol. 18, 767-811 (2000).
Bouwer, H. G., Moors, M. & Hinrichs, D. J. Elimination of the listeriolysin O directed immune response by conservative alteration of the immunodominant listeriolysin O amino acid 91 to 99 epitope. Infect. Immun. 64, 3728-3735 (1996).
Brundage, R. A., Smith, G. A., Camilli, A., Theriot, J. A. & Portnoy, D. A. Expression and phosphorylation of the Listeria monocytogenes ActA protein in mammalian cells. Proc. Nat. Acad. Sci. U.S.A. 90, 11890-11894 (1993).
Busch, D. H., Pilip, I. M., Vijh, S. & Pamer, E. G. Coordinate regulation of complex T-cell populations responding to bacterial infection. Immunity 8, 353-362 (1998).
Edelson, B. T., Cossart, P., & Unanue, E. R. Cutting edge: paradigm revisited: antibody provides resistance to Listeria infection. J. Immunol. 163, 4087-4090 (1999).
Figdor, C. G., de Vries, I. J., Lesterhuis, W. J. & Melief, C. J. M. Dendritic cell immunotherapy: mapping the way. Nat. Med. 10, 475-480 (2004).
Foulds, K. E. et al. Cutting edge: CD4 and CD8 T-cells are intrinsically different in their proliferative responses. J. Immunol. 168, 1528-1532 (2002).
Hamilton, S. E. & Harty, J. T. Quantitation of CD8+ T-cell expansion, memory, and protective immunity after immunization with peptide coated dendritic cells. J. Immunol. 169, 4936-4944 (2002).
Hamilton, S. E., Porter, B. B., Messingham, K. A. N., Badovinac, V. P. & Harty, J. T. MHC class Ia-restricted memory T-cells inhibit expansion of a nonprotective MHC class Ib (H2-M3)-restricted memory response. Nat. Immunol. 5, 159-168 (2004).
Haring, J. S., Corbin, G. A., & Harty J. T. Dynamic Regulation of IFN-65 Signaling in Antigen-Specific CD8+ T-cells Responding to Infection. J. Immunol. (In Press) (2005)
Harrington, L. E., Galvan, M., Baum, L. G., Altman, J. D. & Ahmed, R. Differentiating between memory and effector CD8 T-cells by altered expression of cell surface O-glycans. J. Exp. Med. 191, 1241-1246 (2000).
Harty, J. T. & Bevan, M. J. Specific immunity to Listeria monocytogenes in the absence of IFN gamma, Immunity 3, 109-117 (1995).
Heath, W. R. & Carbone, F. R. Cross-presentation, dendritic cells, tolerance and immunity. Annu. Rev. Immunol. 19, 47-64 (2001).
Hendriks, J., Gravestein, L. A., Tesselaar, K., van Lier, R. A., Schumacher, T. N., & Borst, J. CD27 is required for generation and long-term maintenance of T-cell immunity. Nat. Immunol. 1, 433-440.
Hogquist, K. A. et al. T-cell receptor antagonist peptides induce positive selection. Cell 76, 17-27 (1994).
Huster, K. M. et al. Selective expression of IL-7 receptor on memory T-cells identifies early CD40L-dependent generation of distinct CD8+ memory T-cell subsets. Proc. Natl. Acad. Sci. U.S.A. 101, 5610-5615 (2004).
Kaech, S. M. & Ahmed, R. Memory CD8+ T-cell differentiation: initial antigen encounter triggers a developmental program in naive cells. Nat. Immunol. 2, 415-422 (2001).
Kaech, S. M. et al. Selective expression of the interleukin 7 receptor identifies effector CD8 T-cells that give rise to long-lived memory cells. Nat. Immunol. 4, 1191-1198 (2003).
Kaech, S. M., Hemby, S., Kersh, E. & Ahmed, R. Molecular and functional profiling of memory CD8 T-cell differentiation. Cell 111, 837-851 (2002).
Kaech, S. M., Wherry, E. J. & Ahmed, R. Effector and memory T-cell differentiation: implications for vaccine development. Nat. Rev. Immunol. 2, 251-262. (2002).
Kerksiek, K. M., Busch, D. H., Pilip, I. M., Allen, S. E. & Pamer, E. G. H2-M3restricted T-cells in bacterial infection: rapid primary but diminished memory responses. J. Exp. Med. 190, 195-204 (1999).
Krieg, A. M. CpG motifs: the active ingredient in bacterial extracts? Nat. Med. 9, 831-835 (2003).
Mackaness, G. B. Cellular resistance to infection. J. Exp. Med. 1, 381-406 (1962).
Mercado, R. et al. Early programming of T-cell populations responding to bacterial infection. J. Immunol. 165, 6833-6839 (2000).
Paczesny, S. et al. Expansion of Melanoma-specific Cytolytic CD8+ T-cell Precursors in Patients with Metastatic Melanoma Vaccinated with CD34+ Progenitor-derived Dendritic Cells. J. Exp. Med. 199, 1503-1511 (2004).
Pamer, E. G. Immune responses to Listeria monocytogenes. Nat. Rev. Immunol. 4, 812-823 (2004).
Pernis, A., Gupta, S., Gollob, K. J., Garfein, E., Coffman, R. L., Schindler, C., & Rothman, P. Lack of interferon y receptor beta chain and the prevention of interferon γ signaling in TH1 cells. Science 269:245 (1995).
Roberts, A. D., & Woodland, D. L. Cutting Edge: Effector Memory CD8+ T-cells Play a Prominent Role in Recall Responses to Secondary Viral Infection in the Lung. J. Immunol., 172, 6533 -6537 (2004).
Sallusto, F., Geginat, J. & Lanzavecchia, A. Central Memory and Effector Memory T-cell Subsets: Function, Generation, and Maintenance. Ann. Rev. Immunol. 22, 683-709 (2004).
Schluns, K. S. & Lefrancois, L. Cytokine control of memory T-cell development and survival. Nat. Rev. Immunol. 3, 269-279 (2003).
Schluns, K. S., Kieper, W. C., Jameson, S. C. & Lefrancois, L. Interleukin-7 mediates the homeostasis of naive and memory CD8 T-cells in vivo. Nat. Immunol. 1, 426-432 (2000).
Seder, R. A. & Ahmed, R. Similarities and differences in CD4+ and CD8+ effector and memory T-cell generation. Nat. Immunol. 4, 835-842 (2003).
Shen, H., Miller, J. F., Fan, X., Kolwyck, D., Ahmed, R., Harty, J. T. Compartmentalization of bacterial antigens: differential effects on priming of CD8 T-cells and protective immunity. Cell 92, 535-545 (1998).
Sprent, J. & Surh, C. D. T-cell memory. Ann. Rev. Immunol. 20, 551-579 (2002).
Steinman, R. M., Hawiger, D. & Nussenzweig, M. C. Tolerogenic dendritic cells. Ann. Rev. Immunol. 21, 685-711 (2003).
Takeda, K., Kaisho, T. & Akira, S. Toll-like receptors. Ann. Rev. Immunol. 21, 335-376 (2003).
van Stipdonk, M. J., Lemmens, E. E. & Schoenberger, S. P. Naive CTLs require a single brief period of antigenic stimulation for clonal expansion and differentiation. Nat. Immunol. 2, 423-429 (2001).
Wagner, H. Interactions between bacterial CpG-DNA and TLR9 bridge innate and adaptive immunity. Curr. Opin. Microbiol. 5, 62-69 (2002).
Wherry, E. J. et al. Lineage relationship and protective immunity of memory CD8 T-cell subsets. Nat. Immunol. 4, 225-234 (2003).
Wong, P. & Pamer, E. G. Cutting edge: antigen-independent CD8 T-cell proliferation. J. Immunol. 166, 5864-5868 (2001).
Wong, P., Lara-Tejero, M., Ploss, A., Leiner, I. & Pamer, E. G. Rapid development of T-cell memory. J. Immunol. 172, 7239-7245 (2004).
Woodland, D. L. Jump-starting the immune system: prime-boosting comes of age. Trends Immunol. 25, 98-104 (2004).

### G. SEQUENCES

SEQ ID NO: 1 LLO₉₁₋₉₉
GYKDGNEYI
SEQ ID NO: 2 p60₄₄₉₋₄₅₇
IYVGNGQMI
SEQ ID NO: 3
MIGWII
SEQ ID NO: 4 p60₂₁₇₋₂₂₅
KYGVSVQDI
SEQ ID NO: 5 LLO₉₂F
GFKDGNEYI
SEQ ID NO: 6 OVA₂₅₇₋₂₆₄
SIINFEKL
SEQ ID NO: 7 LCMV NP₁₁₈₋₁₂₆
RPQASGVYM
SEQ ID NO: 8 AH1/AH5 from CT26 colon carcinoma
cells
SPSYAYHQF
SEQ ID NO: 9 Trp1 from B16 melanoma cells
TAYRYHLL
SEQ ID NO: 10 Trp2 from B16 melanoma cells
SVYDFFVWL
SEQ ID NO: 11 LACK from L. donnovani
ICFSPSLEHPIVVSGSWD
SEQ ID NO: 12 Pb9 from P. bergheii
SYIPSAEKI

What is claimed is:

1. A method of producing memory T-cells specific for a target in a subject comprising administering to the subject a mixture comprising an antigen related to the target and a dendritic cell (DC), and administering a booster to the subject less than 6 months from initial antigen contact, and wherein the memory T-cells generated are able to proliferate upon encounter with the booster.

2. The method of claim 1, wherein the booster is administered less than 5 months, 4 months, 3 months, 2 months, 1 month, 4 weeks, 3 weeks, 2 weeks, 13 days, 12 days, 11 days, 10 days, 9 days, 8 days, 7 days, 6 days, 5 days, 4 days, 3 days, 2 days, or 1 day after initial antigen contact.

3. The method of claim 1, wherein the dendritic cells originated from the subject to be vaccinated.

4. The method of claim 1, wherein the dendritic cell is a mature dendritic cell.

5. The method of claim 1, wherein the dendritic cell is a dendritic cell progenitor.

6. The method of claim 1, wherein the antigen is selected from the group of antigens consisting of a peptide, polypeptide, protein, heat-killed pathogen, live attenuated pathogen, or cancer.

7. The method of claim 1, wherein the antigen comprises more than 1 antigen.

8. The method of claim 7, wherein the antigens originate from the same pathogen.

9. The method of claim 7, wherein the antigens originate from heterologous pathogens.

10. The method of claim 1, wherein the antigen is expressed in a vector.

11. The method of claim 1, wherein the antigen is one component in a first vaccine.

12. The method of claim 1, wherein the subject is a mammal, human, mouse, pig, cow, horse, or chicken.

13. The method of claim 1, further comprising administering to the subject a second antigen.

14. The method of claim 13, wherein the first antigen and the second antigen are the same antigen.

15. The method of claim 13, wherein the second antigen is selected from the group of antigens consisting of a peptide, polypeptide, protein, heat-killed pathogen, live attenuated pathogen, or cancer.

16. The method of claim 13, wherein the second antigen is expressed in a vector.

17. The method of claim 13, wherein the second antigen is one component in a second vaccine.

18. The method of claim 13, wherein the second antigen is administered 6, 10, 14, 18, 21, 30, 60, 90, 120, or 180 days after the first antigen.

19. The method of claim 1, whereby the initial DC administration is followed within one week by a booster administration leading to 5-300-fold increases in the number of antigen-specific effector CD8⁺ T-cells within five days after booster administration.

20. The method of claim 19 where the booster administration leads to 3-30-fold increases in the number of memory CD8⁺ T-cells compared to DC vaccination alone and within 30 days after the initial administration.

21. A method of producing protective immunity to a target in a subject, comprising administering a mixture comprising an antigen related to the target and a dendritic cell, wherein the protective immunity is generated within one week, and wherein the subject has sought to achieve protective immunity in an accelerated way.

22. A method of generating a protective amount of central memory T-cells in a subject to multiple antigens comprising mixing dendritic cells with the antigens and administering the mixture to the subject, wherein the protective amount of central memory T-cells are generated more quickly than are generated with the antigen alone.

23. A method of making a vaccine to an antigen comprising mixing a dendritic cell with the antigen and administering the mixture to a subject, wherein the mixture increases the number of T-cells specific to the antigen in the subject.

24. A method of making a vaccine to an antigen comprising mixing dendritic cells with the antigen and administering the mixture to a subject, wherein the mixture accelerates the production of memory T-cells specific to the antigen in the subject.

25. The method of claim 24, wherein the mixture also accelerates the production of the number of effector cells.

26. A method of making a vaccine to an antigen comprising mixing dendritic cells with the antigen and administering the mixture to a subject, wherein the mixture accelerates the production of the effector and memory T-cells specific to the antigen in the subject.

27. A method of making a vaccine specific for a subject in need thereof comprising removing dendritic cells from the subject to be vaccinated, mixing an antigen with the dendritic cells, administering the mixture to the subject.

28. A composition comprising dendritic cells and one or more antigens, wherein the dendritic cells and antigen are in sufficient quantity to induce a protective immune response more quickly and with greater magnitude then antigen alone, wherein the dendritic cells comprise the common MHC alleles for a given population, and wherein the antigen comprise immunodominant peptides corresponding to the MHC alleles.

29. A method of accelerating the production of a protective amount of central memory T-cells in a subject to an antigen comprising mixing dendritic cells with the antigen and administering the mixture to the subject.

30. A method of accelerating the production of a protective amount of central memory T-cells in a subject to multiple antigens comprising mixing dendritic cells with the antigens and administering the mixture to the subject.

31. A method of making a vaccine to an antigen comprising mixing dendritic cells with the antigen and administering the mixture to a subject, wherein the mixture accelerates the transition from effector to memory T-cells specific to the antigen in the subject.


SEQUENCE LISTING (12 sequences)

SEQ ID NO: 1 - protein, 9 aa, Artificial Sequence
  Description of Artificial Sequence; note= synthetic construct
GYKDGNEYI

SEQ ID NO: 2 - protein, 9 aa, Artificial Sequence
  Description of Artificial Sequence; note= synthetic construct
IYVGNGQMI

SEQ ID NO: 3 - protein, 6 aa, Artificial Sequence
  Description of Artificial Sequence; note= synthetic construct
MIGWII

SEQ ID NO: 4 - protein, 9 aa, Artificial Sequence
  Description of Artificial Sequence; note= synthetic construct
KYGVSVQDI

SEQ ID NO: 5 - protein, 9 aa, Artificial Sequence
  Description of Artificial Sequence; note= synthetic construct
GFKDGNEYI

SEQ ID NO: 6 - protein, 8 aa, Artificial Sequence
  Description of Artificial Sequence; note= synthetic construct
SIINFEKL

SEQ ID NO: 7 - protein, 9 aa, Artificial Sequence
  Description of Artificial Sequence; note= synthetic construct
RPQASGVYM

SEQ ID NO: 8 - protein, 9 aa, Artificial Sequence
  Description of Artificial Sequence; note= synthetic construct
SPSYAYHQF

SEQ ID NO: 9 - protein, 8 aa, Artificial Sequence
  Description of Artificial Sequence; note= synthetic construct
TAYRYHLL

SEQ ID NO: 10 - protein, 9 aa, Artificial Sequence
  Description of Artificial Sequence; note= synthetic construct
SVYDFFVWL

SEQ ID NO: 11 - protein, 18 aa, Artificial Sequence
  Description of Artificial Sequence; note= synthetic construct
ICFSPSLEHPIVVSGSWD

SEQ ID NO: 12 - protein, 9 aa, Artificial Sequence
  Description of Artificial Sequence; note= synthetic construct
SYIPSAEKI